SEQUENCE LISTING (36 sequences)

SEQ ID NO: 1 - protein, 192 aa, artemia fransiscana
MALNPWYGGFGGMTDPWSDPFGFGGFGGGMDLDIDRPFRRRMMRRGPDTSRALKELATPGSLRDTADEFQVQLDVGHFLPNEITVKTTDDDILVHGKHDERSDEYGHVQREFRRRYRLPEHVKPESVSSTLSSDGVLTIHAPKTALSSPTERIVPITPAPAVGRIEGGTTGTTTGSTASSTPARTTRSGGAA

SEQ ID NO: 2 - DNA, 709 bp, artemia fransiscana
cggcacgagctcgtgctcaaaatggcacttaacccatggtacggaggatttggtggtatgactgacccatggtctgatccatttggatttggtggcttcggaggtggcatggaccttgatattgacaggcccttccggagaagaatgatgagaagaggtccagataccagcagggctttaaaggagttagctactcctgggtccttgagggacacagctgatgaatttcaagttcagctagatgttggccactttttaccaaacgaaattacagtcaagacaaccgacgatgatattcttgtccatggcaaacatgacgagcgatctgatgaatatggacacgtacaaagagaatttcgacgacgatacagactcccagaacatgtcaaaccagaatctgtgtcatctactttgtcatcagatggtgtcttaactatccatgctccgaaaactgctttaagctcaccaacagaacgtatcgtacccatcacaccagcgccagctgttggaaggattgaagggggaactacaggtactactacaggcagtacagctagttcaactccagcaagaacaacaagatcaggaggtgcagcttaatctgcattcaatatatattcaatgaatcttccggtcttttttctttgtacctttatttttttgtcaataaatctgtatacgaggcaaaaaaaaaaaaaaaaaaaaaaaa

SEQ ID NO: 3 - protein, 165 aa, salmonella typhimurium
MDYQNNVSEERVAEMIWDAVSEGATLKDVHGIPQDMMDGLYAHAYEFYNQGRLDEAETFFRFLCIYDFYNPDYTMGLAAVCQLKKQFQKACDLYAVAFTLLKNDYRPVFFTGQCQLLMRKAAKARQCFELVNERTEDESLRAKALVYLEALKTAETEQHSEQEKE

SEQ ID NO: 4 - DNA, 498 bp, salmonella typhimurium
atggattatcaaaataatgtcagcgaagaacgtgttgcggaaatgatttgggatgccgttagtgaaggcgccacgctaaaagacgttcatgggatccctcaagatatgatggacggtttatatgctcatgcttatgagttttataaccagggacgactggatgaagctgagacgttctttcgtttcttatgcatttatgatttttacaatcccgattacaccatgggactggcggcagtatgccaactgaaaaaacaatttcagaaagcatgtgacctttatgcagtagcgtttacgttacttaaaaatgattatcgccccgttttttttaccgggcagtgtcaattattaatgcgtaaggcagcaaaagccagacagtgttttgaacttgtcaatgaacgtactgaagatgagtctctgcgggcaaaagcgttggtctatctggaggcgctaaaaacggcggagacagagcagcacagtgaacaagaaaaggaataa

SEQ ID NO: 5 - protein, 173 aa, bos taurus
MDIAIQHPWFKRTLGPFYPSRLFDQFFGEGLFEYDLLPFLSSTISPYYRQSLFRTVLDSGISEVRSDRDKFVIFLDVKHFSPEDLTVKVQEDFVEIHGKHNERQDDHGYISREFHRRYRLPSNVDQSALSCSLSADGMLTFSGPKIPSGVDAGHSERAIPVSREEKPSSAPSS

SEQ ID NO: 6 - DNA, 522 bp, bos taurus
atggatatcgccattcagcacccctggttcaaacgcaccctgggccccttctaccccagccggctgttcgaccagttcttcggcgagggcctcttcgagtacgacctgctgcccttcctgtcctccaccatcagcccctactaccgccagtccctcttccgcaccgtgctggactccggcatctctgaggtccgatccgaccgggacaagtttgtcatcttcctggatgtgaagcacttctctcccgaggacctgacggtgaaggtgcaggaggacttcgtggagatccacggcaagcacaacgagcggcaggatgaccatggctacatctcccgcgagttccaccgccgctaccgcctgccttccaacgtggaccagtccgcactctcctgctccctgtccgctgatggcatgctgaccttctctggccccaagatcccatctggcgtggacgccggccacagcgagcgggccatccccgtgtcccgggaggagaagcccagctctgcgccctcgtcctaa

SEQ ID NO: 7 - protein, 175 aa, bos taurus
MDIAIHHPWIRRPFFPFHSPSRLFDQFFGEHLLESDLFPASTSLSPFYLRPPSFLRAPSWIDTGLSEMRLEKDRFSVNLDVKHFSPEELKVKVLGDVIEVHGKHEERQDEHGFISREFHRKYRIPADVDPLAITSSLSSDGVLTVNGPRKQASGPERTIPITREEKPAVTAAPKK

SEQ ID NO: 8 - DNA, 632 bp, bos taurus
atggatatcgccatccaccacccctggatccgccgccccttcttccctttccactctcccagccgcctctttgaccagttttttggcgagcacctgttggagtctgatctcttcccagcttctacttccctgagccccttctaccttcggccgccctcatttctgcgggcacccagctggattgacactggcctctcagagatgcgtctggagaaggacagattctctgtcaacctggatgtgaagcacttctccccagaggaactcaaggtcaaggtgctgggagatgtgattgaggtgcatggcaaacatgaagagcgccaggatgaacatggttttatctcccgggagttccacaggaaataccggatcccagctgacgtggaccctctcgccattacttcatccctgtcctctgatggggtcctcactgtgaatggaccaaggaaacaggcctccggccctgagcgcaccattcccatcacccgtgaagagaagccggctgtcactgcagcccccaagaagtagatgccctttctctaaaccattttttaaaacaagaaaagttccccaccagtgaatgaaaaccttgtgactaatgctgaagctgacggattctctagaaatatcct

SEQ ID NO: 9 - protein, 174 aa, homo sapiens
MGKITLYEDRGFQGRHYECSSDHPNLQPYLSRCNSARVDSGCWMLYEQPNYSGLQYFLRRGDYADHQQWMGLSDSVRSCRLIPHSGSHRIRLYEREDYRGQMIEFTEDCSCLQDRFRFNEIHSLNVLEGSWVLYELSNYRGRQYLLMPGDYRRYQDWGATNARVGSLRRVIDFS

SEQ ID NO: 10 - DNA, 525 bp, homo sapiens
atggggaagatcaccctctacgaggaccggggcttccagggccgccactacgaatgcagcagcgaccaccccaacctgcagccctacttgagccgctgcaactcggcgcgcgtggacagcggctgctggatgctctatgagcagcccaactactcgggcctccagtacttcctgcgccgcggcgactatgccgaccaccagcagtggatgggcctcagcgactcggtccgctcctgccgcctcatcccccactctggctctcacaggatcagactctatgagagggaggactacagaggccagatgatagagttcactgaggactgctcctgtcttcaggaccgcttccgcttcaatgaaatccactccctcaacgtgctggagggctcctgggtcctctacgagctgtccaactaccgaggacggcagtacctgctgatgccaggggactataggcgctaccaggactggggggccacgaatgccagagtgggctctctgaggagagtcatagatttctcctga

SEQ ID NO: 11 - protein, 14 aa, Artificial Sequence
  enzyme substrate
KHLGGGALGGGAKE

SEQ ID NO: 12 - protein, 4 aa, Artificial Sequence
  enzyme substrate
AAPV

SEQ ID NO: 13 - protein, 7 aa, Artificial Sequence
  enzyme substrate
PLGLDAR

SEQ ID NO: 14 - protein, 179 aa, Artificial Sequence
  Pep/Lew/alpha-crystallin chimeric protein
MVVALLRDIAIQHPWFKRTLGPFYPSRLFEQFFGEGLFEYDLLPFLSSTISPYYRQSLFRTVLDSGISEVRSDRDKFVIFLDVKHFSPEDLTVKVQEDFVEIHGKHNERQDDHGYISREFHRRYRLPSNVDQSALSCSLSADGMLTFSGPKIPSGVDAGHSERAIPVSREEKPSSAPSS

SEQ ID NO: 15 - protein, 173 aa, Artificial Sequence
  synthetic heat stable crystallin
MYKMYMYEEHYMYYHHYEMYYEHMYMYMYMYHMYYYHMEYYMWMMYEYMYYYMYYMMHHYEYYEHYYWMYMYEYMHYMHMMMHYYYHHMHMYEHEEYHYYMMEMYEEMYMMYEHMHMYEMHYMYMMEYYWMMYEMYYYHYHYYMMMMYEYHHYYEWYMYYYHMYYMHHMMEMY

SEQ ID NO: 16 - DNA, 540 bp, Artificial Sequence
  Pep/Lew/alpha-crystallin chimeric protein
atggtagtagcactactacgagatatcgccattcagcacccctggttcaaacgcaccctgggccccttctaccccagccggctgttcgaacagttcttcggcgagggcctcttcgagtacgacctgctgcccttcctgtcctccaccatcagcccctactaccgccagtccctcttccgcaccgtgctggactccggcatctctgaggtccgatccgaccgggacaagtttgtcatcttcctggatgtgaagcacttctctcccgaggacctgacggtgaaggtgcaggaggacttcgtggagatccacggcaagcacaacgagcggcaggatgaccatggctacatctcccgcgagttccaccgccgctaccgcctgccttccaacgtggaccagtccgcactctcctgctccctgtccgctgatggcatgctgaccttctctggccccaagatcccatctggcgtggacgccggccacagcgagcgggccatccccgtgtcccgggaggagaagcccagctctgcgccctcgtcctaa

SEQ ID NO: 17 - protein, 173 aa, homo sapiens
MDVTIQHPWFKRTLGPFYPSRLFDQFFGEGLFEYDLLPFLSSTISPYYRQSLFRTVLDSGISEVRSDRDKFVIFLDVKHFSPEDLTVKVQDDFVEIHGKHNERQDDHGYISREFHRRYRLPSNVDQSALSCSLSADGMLTFCGPKIQTGLDATHAERAIPVSREEKPTSAPSS

SEQ ID NO: 18 - DNA, 522 bp, homo sapiens
atggacgtgaccatccagcacccctggttcaagcgcaccctggggcccttctaccccagccggctgttcgaccagtttttcggcgagggcctttttgagtatgacctgctgcccttcctgtcgtccaccatcagcccctactaccgccagtccctcttccgcaccgtgctggactccggcatctctgaggttcgatccgaccgggacaagttcgtcatcttcctcgatgtgaagcacttctccccggaggacctcaccgtgaaggtgcaggacgactttgtggagatccacggaaagcacaacgagcgccaggacgaccacggctacatttcccgtgagttccaccgccgctaccgcctgccgtccaacgtggaccagtcggccctctcttgctccctgtctgccgatggcatgctgaccttctgtggccccaagatccagactggcctggatgccacccacgccgagcgagccatccccgtgtcgcgggaggagaagcccacctcggctccctcgtcctaa

SEQ ID NO: 19 - protein, 175 aa, homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

SEQ ID NO: 20 - DNA, 528 bp, homo sapiens
atggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacaggaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatag

SEQ ID NO: 21 - protein, 215 aa, homo sapiens
METQAEQQELETLPTTKMAQTNPTPGSLGPWKITIYDQENFQGKRMEFTSSCPNVSERSFDNVRSLKVESGAWIGYEHTSFCGQQFILERGEYPRWDAWSGSNAYHIERLMSFRPICSANHKESKMTIFEKENFIGRQWEISDDYPSLQAMGWFNNEVGSMKIQSGAWVCYQYPGYRGYQYILECDHHGGDYKHWREWGSHAQTSQIQSIRRIQQ

SEQ ID NO: 22 - DNA, 648 bp, homo sapiens
atggagacccaggctgagcagcaggagctggaaacccttccaaccaccaagatggctcagaccaaccctacgccggggtccctggggccatggaagataaccatctatgatcaggagaactttcagggcaagaggatggagttcaccagctcctgtccaaatgtctctgagcgcagttttgataatgtccggtccctgaaggtggaaagtggcgcctggattggttatgagcataccagcttctgtgggcaacagtttatcctggagagaggagaataccctcgctgggatgcctggagtgggagtaatgcctaccacattgagcgtctcatgtccttccgccccatctgttcagctaatcataaggagtctaagatgaccatctttgagaaggaaaactttattggacgccagtgggagatctctgacgactacccctccttgcaagccatgggctggttcaacaacgaagtcggctccatgaagatacaaagtggggcctgggtttgctaccaatatcctggatatcgtgggtatcagtatatcttggaatgtgaccatcatggaggagactataaacattggagagagtggggctctcatgcccagacttcgcagatccaatcgattcgccgaatccaacagtag

SEQ ID NO: 23 - protein, 447 aa, unknown
  chicken
MATEGDKLLGGRFVGSTDPIMEILSSSISTEQRLTEVDIQASMAYAKALEKASILTKTELEKILSGLEKISEESSKGVLVMTQSDEDIQTAIERRLKELIGDIAGKLQTGRSRNEQVLTDLKLLLKSSTSVISTHLLQLIKTLVERAAIEIDIIMPGYTHLQKALPIRWSQFLLSHAVALTRDSERLGEVKKRITVLPLGSGALAGNPLEIDRELLRSELDMTSITLNSIDAISERDFVVELISVATLLMIHLSKLAEDLIIFSTTEFGFVTLFDAYSTGSSLLPQKKNPDSLELIRSKAGRVFGRLAAILMVLKGIPSTFSKDLQEDKEAVLDVVDTLTAVLQAATEVISTLQVNKENMEKALTPELLSTDLALYLVRKGMPIRQAQTASGKAVHLAETKGITINNLTLEDLKSISPLFASDVSQVFSVVNSVEQYTAVGGTAKAA

SEQ ID NO: 24 - DNA, 1344 bp, unknown
  chicken
atggcaaccgagggggataaacttttgggaggaagatttgttggaagcacagatcccatcatggagattctcagctcttctatatccactgagcagagactgactgaagttgatatccaagcaagcatggcttatgccaaagccttggagaaggctagcatcctgactaaaactgagctggagaagatcctgagtggcctggaaaagatctctgaggaatcatctaagggagtccttgtaatgacccaaagtgatgaagatatccagactgccattgaacgcagactgaaggagctgattggggatatagctggaaagctgcagactggaagaagcaggaatgaacaggttttgactgatctgaaactgctcctgaagagttccacttctgtcatctccactcatctgctgcagctcatcaagaccctggtggagcgcgctgccatagaaattgatattatcatgcctggctacacccacctgcagaaagctctgcccatcagatggagccagttcctgctcagccatgctgttgcactgacccgtgattctgagcgcctgggagaggtgaagaaaaggatcactgtcttgcctctgggaagtggtgctctggctggcaacccactggaaattgatagagagcttctgcgtagcgaactggacatgacttccatcaccctgaacagcatagacgccatcagtgagagagactttgtggtggaattaatctctgttgccaccctgctgatgattcaccttagcaagctggctgaagatctcatcatcttcagcaccactgaatttggctttgtgaccctctttgatgcctacagcactggcagcagcctgttgcctcagaagaagaaccctgatagcctagaactgatccgcagcaaagctggtcgtgtgtttggacggttggctgctattctcatggttctcaaaggaattccaagcaccttcagcaaggatctgcaggaggacaaggaagctgtccttgatgttgtggacactctgactgctgtgctccaggctgccactgaagtgatttctaccctccaggtcaacaaggagaacatggagaaggctctgacccctgagttgctgtctactgatctggctctctacttggttcgtaaaggaatgccaatcagacaagcccaaactgcttctgggaaggccgtccaccttgctgagactaaaggcatcaccatcaataatctcaccctagaggacctgaagagcatcagccccctgtttgccagcgatgtctcccaggtcttcagcgttgtcaacagtgtggagcagtacactgccgtgggtggtactgccaaggcagcgtga

SEQ ID NO: 25 - protein, 12 aa, Artificial Sequence
  enzyme substrate
KAAHKSALKSAE

SEQ ID NO: 26 - protein, 14 aa, Artificial Sequence
  enzyme substrate
KHLGGGALGGGAKE

SEQ ID NO: 27 - protein, 20 aa, Artificial Sequence
  protective protein
MGKITLYEDRGFQGRHYECS

SEQ ID NO: 28 - protein, 78 aa, Artificial Sequence
  alpha-crystallin/calcium binding domain of calmodulin chimeric protein
MGKITLYEDRGFQGRHYECSSFDKDGNGYISAAELRHVMTNLGEKLTDEEVDEMIREADIDGDGQVNYEEFVQMMTAK

SEQ ID NO: 29 - DNA, 27 bp, Artificial Sequence
  primer
cccaagcttggggccaggaacacacag

SEQ ID NO: 30 - DNA, 27 bp, Artificial Sequence
  primer
cccaagcttgggtttgacaaggatggc

SEQ ID NO: 31 - DNA, 27 bp, Artificial Sequence
  primer
cgcggatccgcgtcattttgcagtcat

SEQ ID NO: 32 - protein, 30 aa, Artificial Sequence
  heat stable peptide
MYKMYMYEEHYMYYHHYEMYYEHMYMYMYM

SEQ ID NO: 33 - DNA, 27 bp, Artificial Sequence
  primer
ctagctagctagatggggaagatcacc

SEQ ID NO: 34 - protein, 18 aa, Artificial Sequence
  truncated alpha-crystallin C domain
SGPKIPSREEKPSSAPSS

SEQ ID NO: 35 - protein, 22 aa, Artificial Sequence
  chimeric alpha-crystallin C domain
MYKMYMYEEHSREEKPSSAPSS

SEQ ID NO: 36 - protein, 11 aa, Artificial Sequence
  peptide substrate
KVSRRRRRGGD

(19) United States
(12) Patent Application Publication
(10) Pub. No.: US 2006/0205646 A1
(43) Pub. Date: Sep. 14, 2006

(54) Methods for increasing cell and tissue viability

(76) Inventors: Mitchell C. Sanders, West Boylston, MA (US); Diane L. Ellis-Busby, Lancaster, MA (US); Shite Sebastian, Somerville, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/271,158

(22) Filed: Nov. 11, 2005

Related U.S. Application Data
(63) Continuation of application No. PCT/US04/14920, filed on May 12, 2004.
(60) Provisional application No. 60/537,814, filed on Jan. 21, 2004.
(60) Provisional application No. 60/469,869, filed on May 12, 2003.

(51) Int. Cl. A61K 38/16

(52) U.S. Cl. 514/12

(57) ABSTRACT

The present invention features methods of increasing cell or tissue viability by administering to the cell or tissue a protective protein. The invention also features methods of treating a condition characterized by cell or tissue damage in a subject by administering to the subject a protective protein. Also included are chimeric proteins as well as methods of inhibiting proteolysis of a cationic antimicrobial peptide in a cell or tissue including contacting the cell or tissue with a protective protein, chimeric protein that includes the protective protein, or a biologically active fragment, variant, or derivative thereof.

### RELATED APPLICATIONS

This application is a continuation of International Application No. PCT/US2004/014920, which designated the United States and was filed May 12, 2004, published in English, which claims the benefit of U.S. Provisional Application No. 60/537,814, filed Jan. 21, 2004, and U.S. Provisional Application No. 60/469,869, filed May 12, 2003. The entire teachings of these applications are incorporated herein by reference.

### BACKGROUND OF THE INVENTION

Tissue damage is caused by a substantive loss of tissue due to apoptosis or tissue necrosis, or due to an injury in which tissue is destroyed. Cell and tissue damage can occur in a number of different acute and chronic diseases and conditions. The degree to which cell or tissue damage occurs is mediated by many factors, including the type of disease, the level of inflammation associated with the disease, the location of the cell or tissue damage, age of the person with the disease, immunological status of the person with the disease, the vascular sufficiency of the tissue, and whether the-tissue is infected. Given proper medical treatment, cell and tissue damage can be repaired, and the disease can be maintained or cured. Alternatively, when the level of cell or tissue damage is high, the result is destruction of the tissue.

While it is important to treat any condition in which the potential for cell or tissue damage exists immediately (e.g., an acute wound), it is essential that certain conditions be treated before they become chronic conditions. Chronic diseases are a challenge to the patient, the health care professional, and to the health care system. They significantly impair the quality of life for millions of people in the United States. Intensive treatment is required with a high cost to society in terms of lost productivity and health care dollars. The management of chronic diseases can place an enormous strain on health care resources. Diseases or conditions, for example, wounds that were once acute but have progressed to chronic often do so because the diseases can not be controlled or treated with known therapies. Therefore, there is a need for improved therapies for treating chronic diseases and conditions characterized by cell and tissue damage.

### SUMMARY OF THE INVENTION

It has been discovered that a group of proteins called “protective proteins” (e.g., PROTECTOR PROTEINS™ brand of protective proteins, available from Expressive Constructs, Inc., Worcester, Mass.) can be used to inhibit cell and tissue damage and to increase cell viability. Such protective proteins can be used to treat conditions characterized by cell or tissue damage.

The protective proteins of the present invention are typically from a class of small chaperone proteins isolated from a variety of sources (e.g., crustaceans, mammals, and bacteria). In particular, the protective proteins of the present invention encompass, for example, p26, SicA, and crystallin proteins, biologically active fragments thereof, chimeric proteins, variants, analogs, and derivatives of the protective proteins. In another embodiment, the crystallin protein is selected from the group consisting of α-A-crystallin, α-B-crystallin, and γ-D-crystallin. In another embodiment, the protective protein is a chimeric protein comprising, p26, SicA, or a crystallin protein or proteins.

Specifically encompassed in the present invention are novel, crystallin/small molecule chaperone-type proteins/peptides that are heat-stable. Such proteins/peptides (referred to herein as protective proteins) are heat stable (e.g., retain their biological activity of protection against cell and tissue damage) even after multiple rounds of autoclaving (e.g., one, two, three, or more than three 25 minute rounds of autoclaving at 120° C.). These heat-stable proteins/peptides are also referred to herein as heat gamma (γ) proteins or HG proteins.

In one aspect, the invention features a method of inhibiting cell or tissue damage, comprising contacting the cell or tissue with a protective protein or an active fragment, variant or derivative thereof, or a chimeric protein comprising the protective protein or an active fragment, variant or derivative thereof. The contact inhibits the cell or tissue damage. In one embodiment, the cell or tissue damage is pathogen-induced.

In another aspect, the invention features a method of increasing cell or tissue viability, comprising contacting the cell or tissue with a protective protein or an active fragment, variant or derivative thereof, or with a chimeric protein comprising the protective protein or an active fragment, variant or derivative thereof. The contact increases viability of the cell or tissue.

In another aspect, the invention features a method of treating a condition characterized by cell or tissue damage in a subject, comprising administering to the subject a protective protein or an active fragment, variant or derivative thereof, or a chimeric protein comprising the protective protein or an active fragment, variant or derivative thereof. In one embodiment, the cell or tissue damage is pathogen-induced. In another embodiment, the condition is selected from the group consisting of a wound (e.g., a chronic or acute wound), corneal damage (e.g., microbial keratitis), meningitis, apoptosis, necrosis, bums, scar prevention, inflammation, damage to a heart valve, varicose veins, an irritation (e.g., rash), incisions, for example, made by surgery or other invasive procedures (e.g., insertion of a stent, catheter, medical device, or drug delivery system), cosmetic applications (e.g., bums, irritations, or rashes), ulcers (e.g., diabetic ulcers, pressure ulcers, venous leg ulcers, corneal ulcers, and peptic ulcers), valve stenosis, gingivitis, endocarditis, and calcification of cardiac tissue.

In another aspect, the invention features a method of enhancing wound healing in a subject, comprising administering to the subject a protective protein or an active fragment, variant or derivative thereof, or a chimeric protein comprising the protective protein or an active fragment, variant or derivative thereof. In one embodiment, the wound is an acute wound. In another embodiment, the wound is a chronic wound.

In another embodiment, the invention features a method of inhibiting protease activity in a cell or tissue, comprising contacting the cell or tissue with a protective protein or an active fragment, variant or derivative thereof, or a chimeric protein comprising the protective protein or an active fragment, variant or derivative thereof. In one embodiment, the protease is a matrix metalloprotease or an elastase.

In another aspect, the invention features a method of inhibiting a protease secreted by a pathogen, comprising contacting a protein secreted by the pathogen with a protective protein or an active fragment, variant or derivative thereof, or a chimeric protein comprising the protective protein or an active fragment, variant or derivative thereof. In one embodiment, the pathogen is of the genus Pseudomonas (e.g., P. aeruginosa), Staphylococcus, and/or Serratia.

In another aspect, the invention features a method of inhibiting proteolysis of a cationic antimicrobial peptide in a cell or tissue comprising contacting the cell or tissue with a protective protein or an active fragment, variant or derivative thereof, or a chimeric protein comprising the protective protein or an active fragment, variant or derivative thereof. The cationic antimicrobial peptide can be, for example, a defensin, a cathelicidin, or a thrombocidin.

In yet another aspect, the invention features methods of inhibiting the virulence of a pathogen comprising contacting an agent or substance released by the pathogen with a protective protein or an active fragment, variant or derivative thereof, or a chimeric protein comprising the protective protein or an active fragment, variant or derivative thereof.

In another aspect, the invention features a chimeric protein comprising Pepstatin A, Leupeptin, and an alpha-A-crystallin polypeptide. In one embodiment, the chimeric protein comprises or consists of the sequence of SEQ ID NO: 14.

In another aspect, the invention features a high heat stable crystallin protein. In one embodiment, the high heat stable protein comprises or consists of the sequence of SEQ ID NO: 15. In another embodiment the high heat stable protein is a protective protein.

The protective proteins of the present invention can be combined with a pharmaceutically acceptable carrier to form a protective protein composition. The protective proteins of the present invention can also be attached to a wound, dressing, or bandage. Optionally, the protective proteins are coated, absorbed into or onto, or in some other way attached, or in close contact with, a material that comes into contact with a wound, for example, sutures, wound drainage materials, catheters, etc. In another embodiment, the protective protein is part of a wound cleaning or protecting solution, cream, ointment, or an aerosol or spray. In still another embodiment, the protective protein can be coated, absorbed into or onto, or in close contact with suitable biologically inert materials, such as plastics. In a particular embodiment of the present invention the inert material can be a contact lens. In another embodiment, the protective protein can be part of a solution that can be used to prevent cell damage. Such solutions can be a washing solution, or rinsing solution, for example, an eye wash solution, a contact lens cleaning solution, eye drops, or ointment.

In a further embodiment, the present invention provides methods and compositions comprising a protective protein to alleviate one or more factors that contribute to the degradation of heart valves (e.g., mechanical valves or tissue valves). For example, the protective proteins can be administered to individuals to prevent, slow, halt, and/or reverse the degradation of heart valves, or can be used to coat mechanical heart valves or heart tissue valves that are to be inserted into a subject.

In yet another embodiment, the present invention provides methods and compositions which alleviate one or more factors that contribute to periodontal disease (e.g., gingivitis or periodontitis). For example, the protective proteins can be administered to individuals, systemically or locally, to prevent, slow, halt, and/or reverse one or more periodontal diseases.

### BRIEF DESCRIPTION OF THE DRAWINGS

FIG. 1 is a schematic representation of the recovery phase in normal and chronic wounds.

FIG. 2 is a graph showing the specificity of P. aeruginosa for the substrate PALA1. Supernatant from P. aeruginosa, S. marcescens, S. aureus, S. epidermidis, S. salivarius, S. pyogenes, E. faecalis, and E. coli bacterial cultures were reacted with the substrate PALA1, and cleavage of PALA1 (relative fluorescence) was followed at 485 nm on a fluorometric plate reader over time (seconds).

FIG. 3 is a graph of showing the inhibition of protease activity of a bacterial protease from P. aeruginosa PA14 on PALA1 peptide using γ-D-crystallin over time (seconds). The positive control was protease substrate with PA14 supernatant and the negative control was protease substrate with buffer.

FIG. 4 is a graph of the inhibition of MMP activity by γ-D-crystallin or EDTA over time, as measured by relative fluorescence of an MMP substrate.

FIG. 5 is a histogram of cell viability measured in tissue culture cells (normal human astrocytes, NHA) exposed to buffered saline (PBS 0.5M NaCl), bovine serum albumin (BSA), or p26 incubated at elevated temperatures. The surviving cell count is indicated.

FIG. 6 illustrates a graph of relative healing scores on the third day of a wound healing test.

FIG. 7 is a graph of the cleavage of PAPA1 peptide or PALA1 peptide in uninfected corneal cultures or in a P. aeruginosa infected corneal culture (as measured by relative fluorescence of the peptides). Negative control was phosphate buffered saline (PBS).

FIG. 8 is a graph showing the specificity of P. aeruginosa for the substrate PAPA 1. Supernatant from P. aeruginosa, S. marcescens, S. aureus, S. epidermidis, S. salivarius, S. pyogenes, E. faecalis, and E. coli bacterial cultures were reacted with the substrate PAPA1, and cleavage of PAPA1 (relative fluorescence) was followed at 485 nm on a fluorometric plate reader over time (seconds).

FIG. 9A is the amino acid sequence of the p26 protein from Artemia franciscana (SEQ ID NO: 1).

FIG. 9B is the nucleotide sequence of the p26 nucleic acid molecule from Artemia franciscana (SEQ ID NO: 2).

FIG. 9C is the amino acid sequence of the SicA protein from Salmonella typhimurium (SEQ ID NO: 3).

FIG. 9D is the nucleotide sequence of the SicA nucleic acid molecule from Salmonella typhimurium (SEQ ID NO: 4).

FIG. 9E is the amino acid sequence of the α-A-crystallin protein from Bos taurus (SEQ ID NO: 5).

FIG. 9F is the nucleotide sequence of the α-A-crystallin nucleic acid molecule from Bos taurus (SEQ ID NO: 6).

FIG. 9G is the amino acid sequence of the α-B-crystallin protein from Bos taurus (SEQ ID NO: 7).

FIG. 9H is the nucleotide sequence of the α-B-crystallin nucleic acid molecule from Bos taurus (SEQ ID NO: 8).

FIG. 91 is the amino acid sequence of the γ-D-crystallin protein from Homo sapiens (SEQ ID NO: 9).

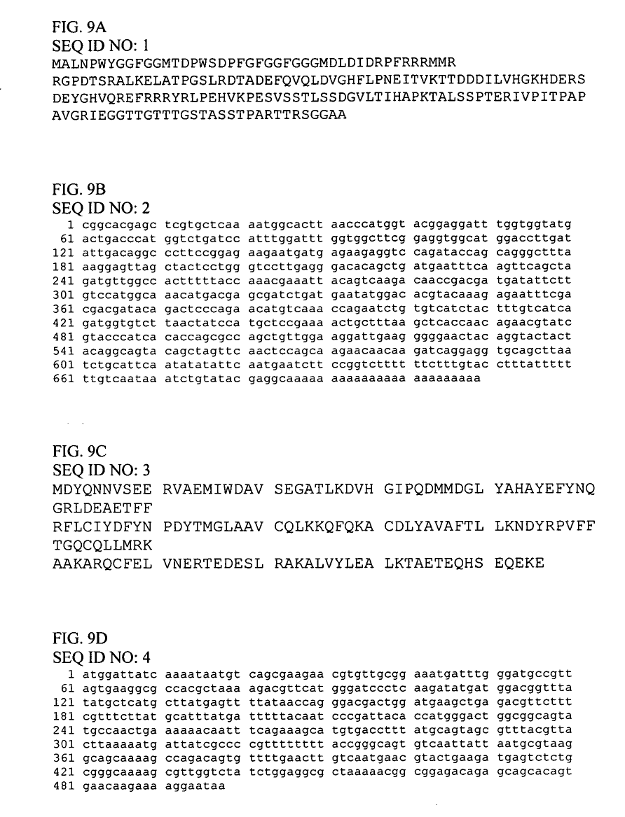

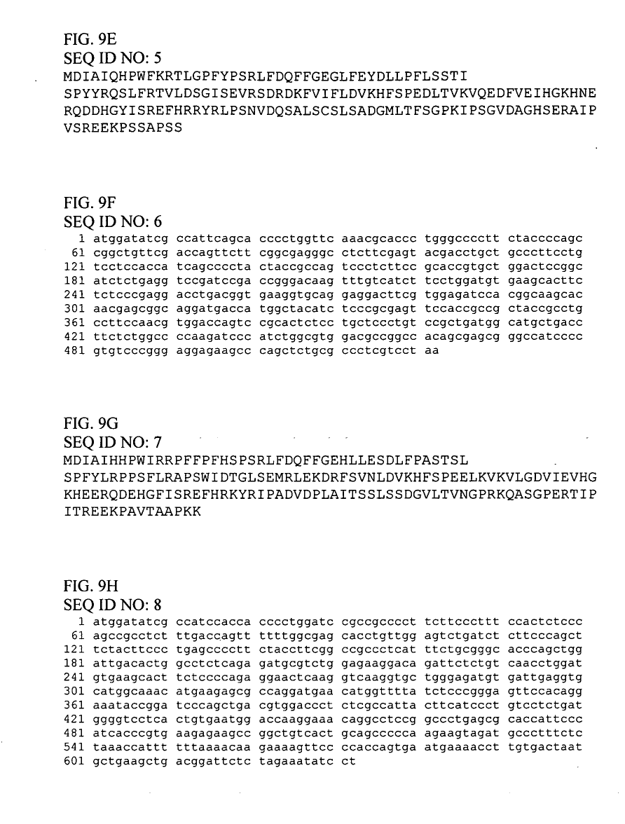

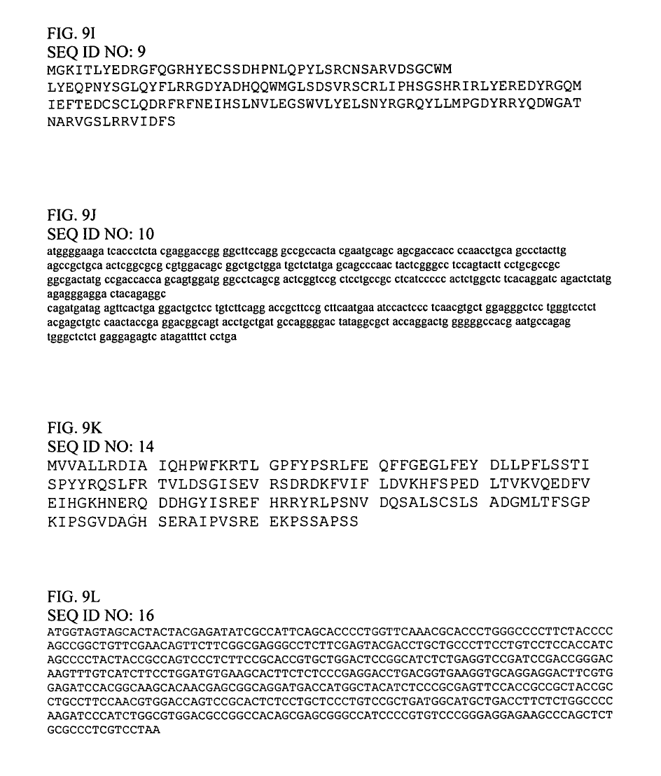

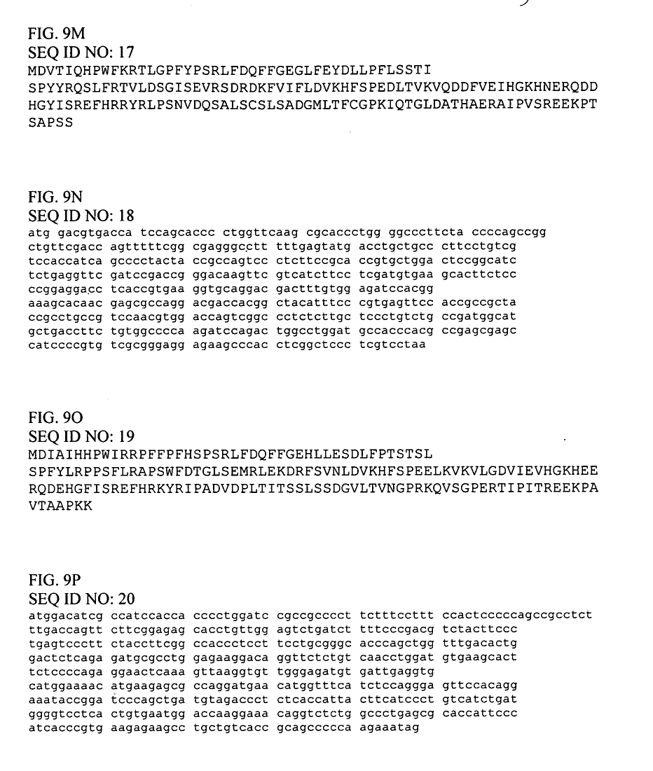

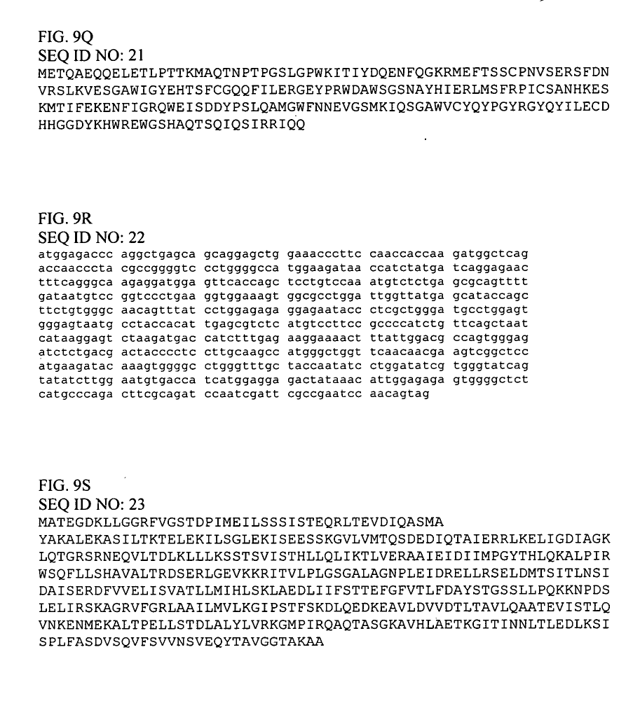

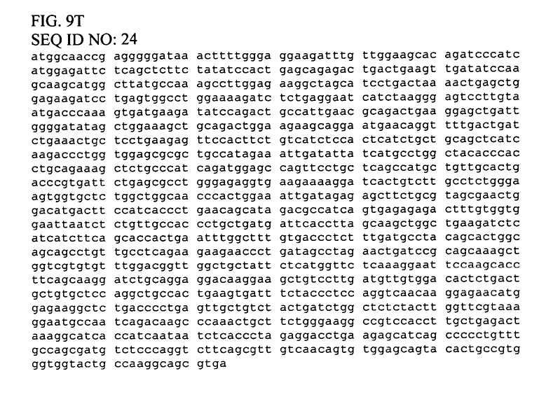

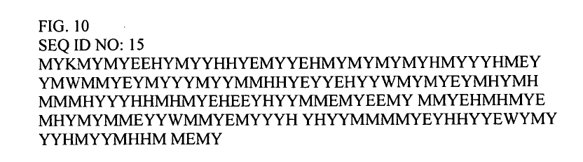

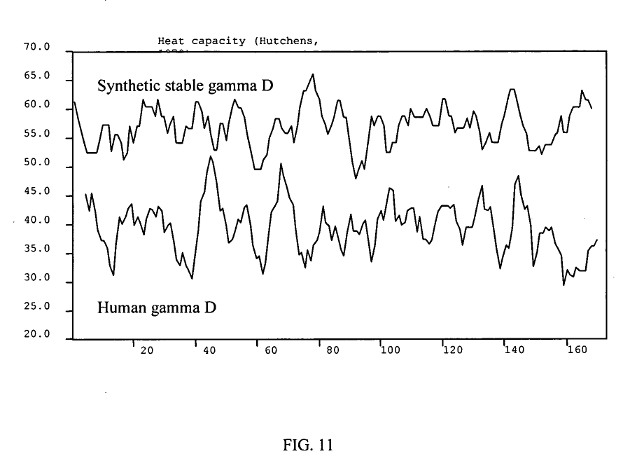

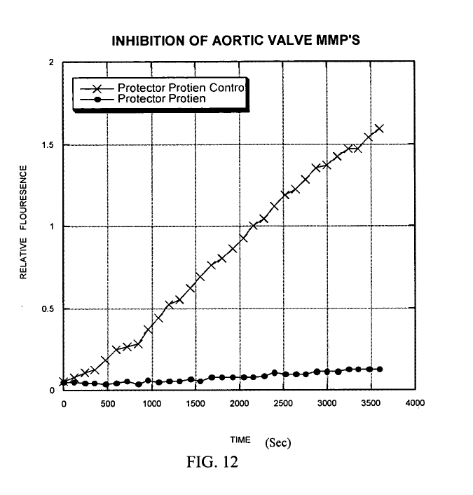

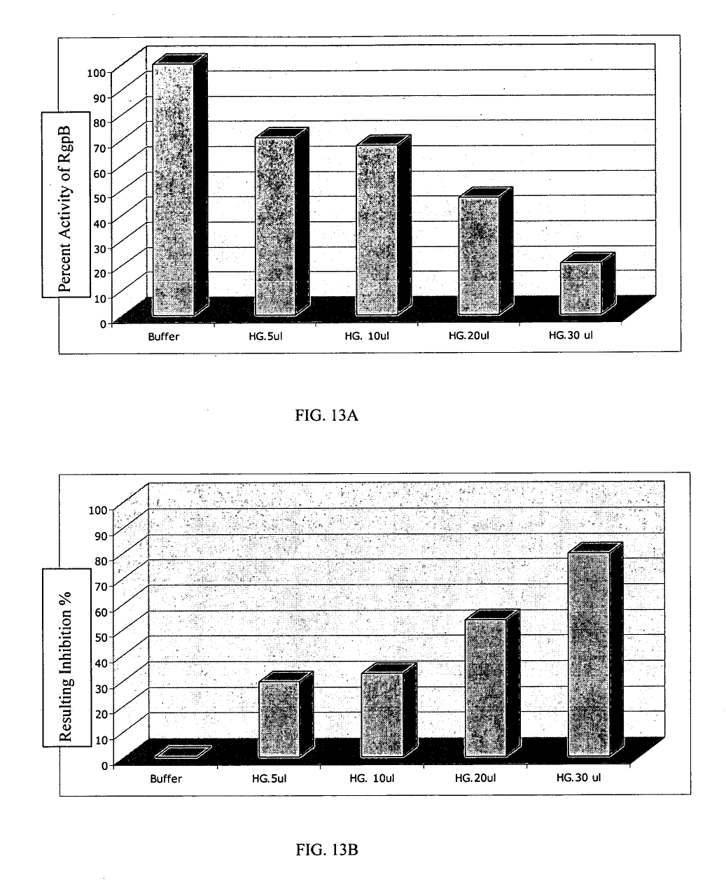

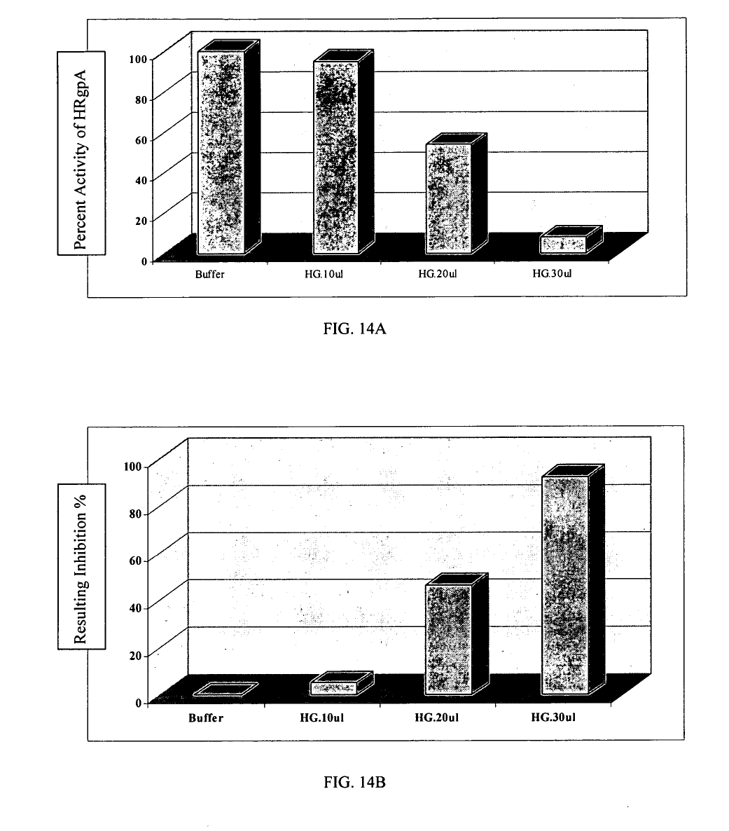

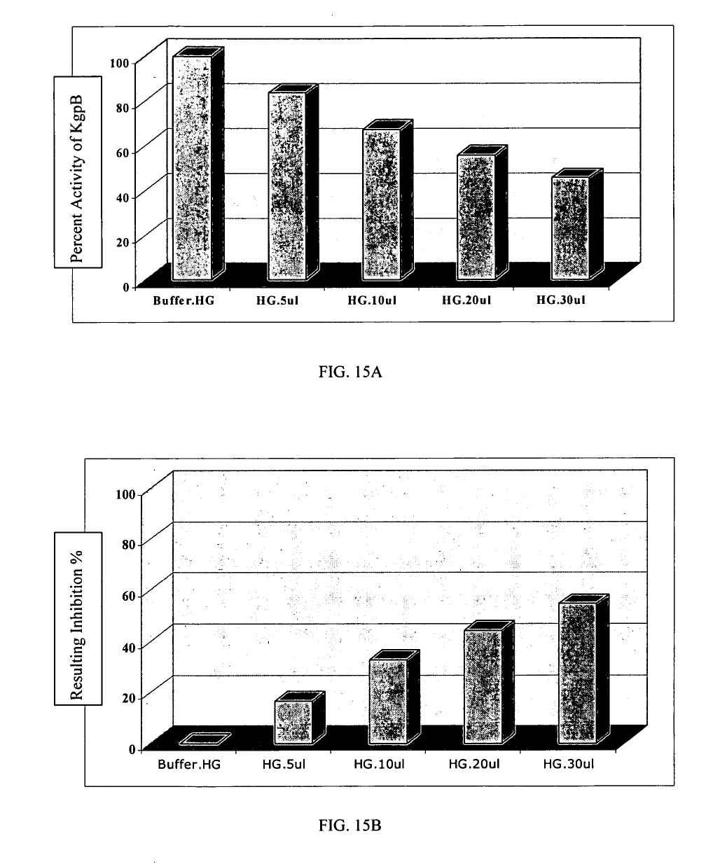

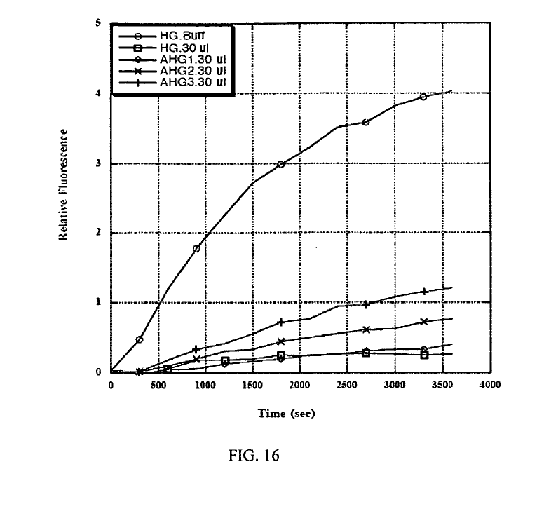

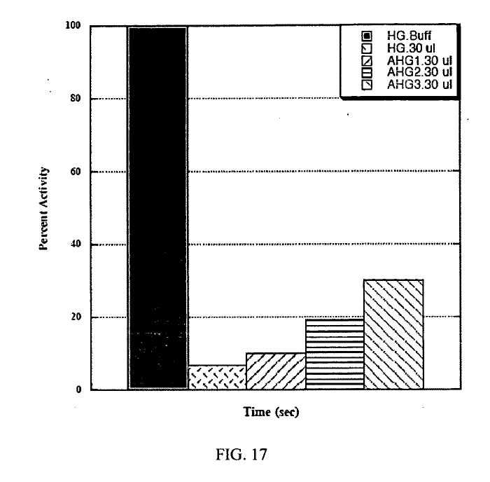

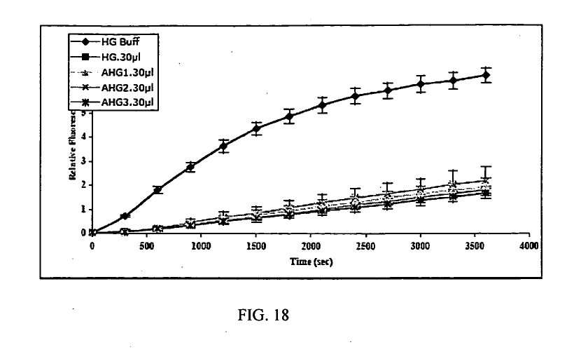

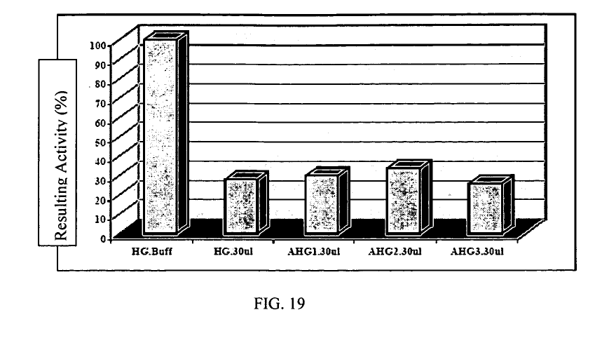

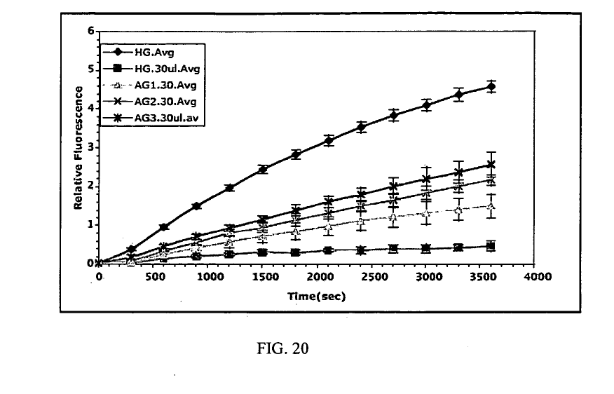

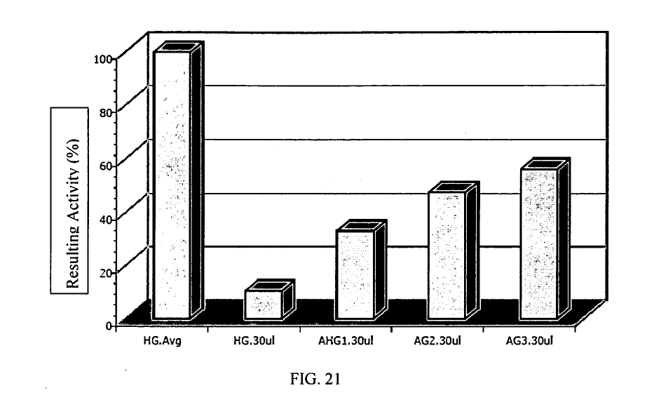

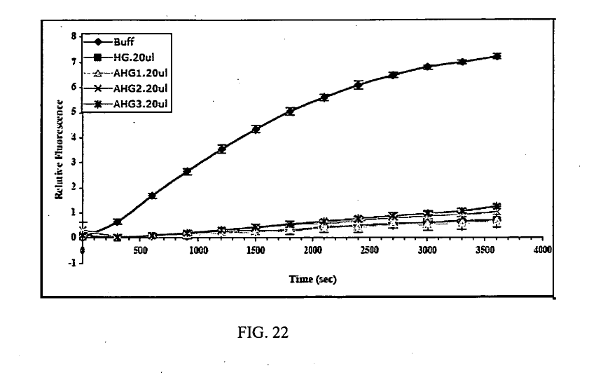

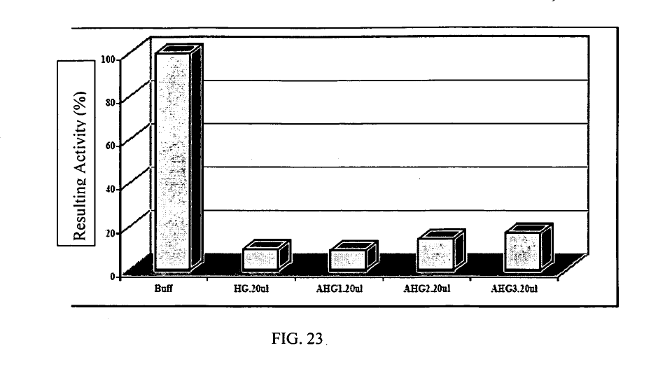

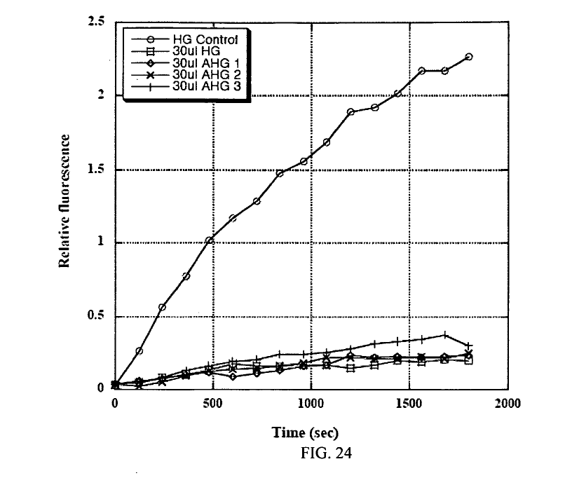

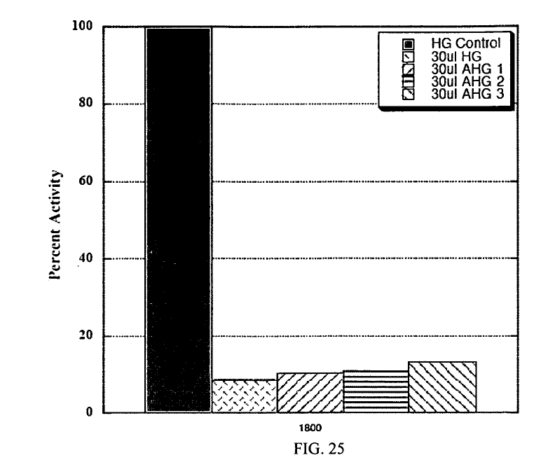

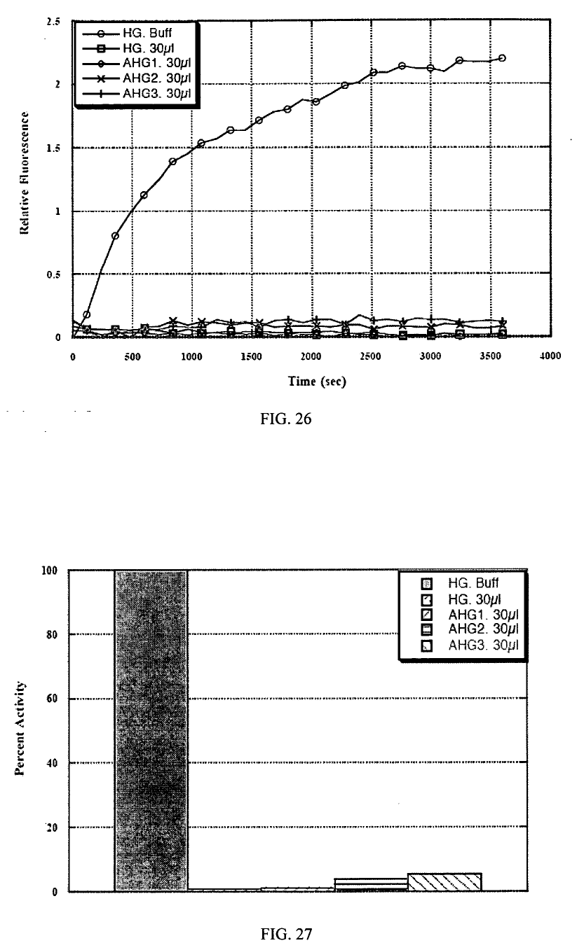

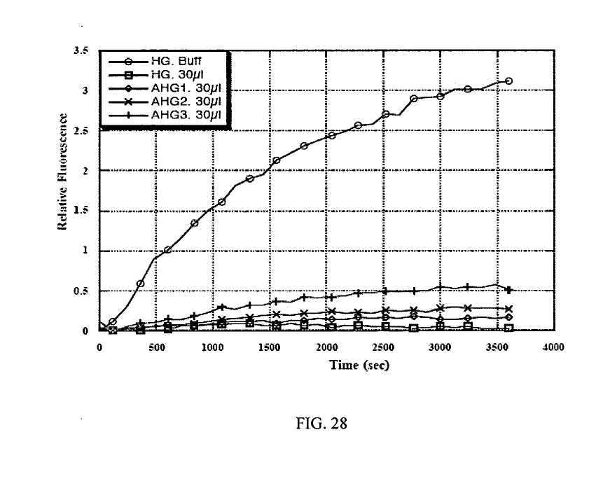

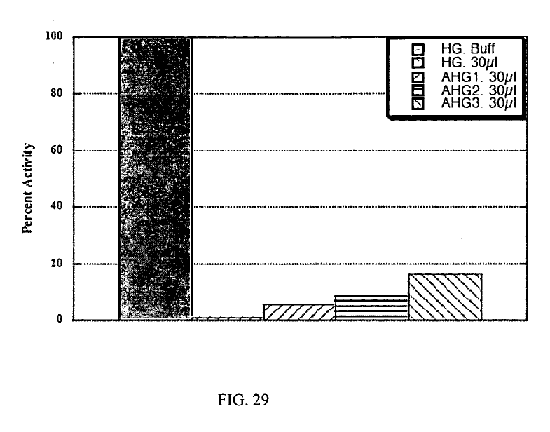

FIG. 9J is the nucleotide sequence of the γ-D-crystallin nucleic acid molecule from Homo sapiens (SEQ ID NO: 10).

FIG. 9K is the amino acid sequence of a Pep/Leu/α-A-crystallin chimeric protein (SEQ ID NO: 14).

FIG. 9L is the nucleotide sequence encoding a Pep/Leu/α-A-crystallin chimeric protein (SEQ ID NO: 16).

FIG. 9M is the amino acid sequence of the α-A-crystallin protein from Homo sapiens (SEQ ID NO: 17).

FIG. 9N is the nucleotide sequence of the α-A-crystallin nucleic acid molecule from Homo sapiens (SEQ ID NO: 18).

FIG. 9O is the amino acid sequence of the α-B-crystallin protein from Homo sapiens (SEQ ID NO: 19).

FIG. 9P is the nucleotide sequence of the α-B-crystallin nucleic acid molecule from Homo sapiens (SEQ ID NO: 20).

FIG. 9Q is the amino acid sequence of the β-A1 -crystallin protein from Homo sapiens (SEQ ID NO: 21).

FIG. 9R is the nucleotide sequence of the β-A1 -crystallin nucleic acid molecule from Homo sapiens (SEQ ID NO: 22).

FIG. 9S is the amino acid sequence of the Δ-crystallin protein from chicken (SEQ ID NO: 23).

FIG. 9T is the nucleotide sequence of the Δ-crystallin nucleic acid molecule from chicken (SEQ ID NO: 24).

FIG. 10 is the amino acid sequence of a synthetic high heat stable crystallin protein (SEQ ID NO: 15).

FIG. 11 is a graph showing the heat capacity differential between native human γ-D-crystallin (SEQ ID NO: 9) and the high heat stable crystallin protein (SEQ ID NO: 15) over the lengths of the proteins.

FIG. 12 illustrates the inhibition of MMP activity from controls and samples containing heart valve extract.

FIG. 13A includes a bar graph illustrating the resulting activity of RgpB in the presence of incrementally larger concentrations of heat gamma.

FIG. 13B includes a bar graph illustrating the resulting inhibition for the same samples as in FIG. 13A.

FIG. 14A includes a bar graph illustrating the relative activity of a protein HRgpA in the presence of incrementally larger concentrations of heat gamma.

FIG. 14B includes a bar graph illustrating the resulting inhibition for the same samples as in FIG. 14A.

FIG. 15A includes a bar graph illustrating the resulting activity of KGP in the presence of incrementally larger concentrations of heat gamma.

FIG. 15B includes a bar graph illustrating the resulting inhibition for the same samples as in FIG. 15A.

FIG. 16 illustrates a graph of the relative fluorescence of a recombinant isolate of Pseudomonas digesting a FRET peptide in the absence (HG buffer) or presence of heat gamma autoclaved 1, 2 or 3 times (AHG1, AHG2, and AHG3 respectively).

FIG. 17 illustrates a graph of the resulting activity for the three samples illustrated of FIG. 16.

FIG. 18 illustrates a graph of the relative fluorescence of a clinical isolate of S. aureus digesting a FRET peptide in the absence (HG buffer) or presence of heat gamma autoclaved 1, 2 or 3 times (AHG1, AHG2, and AHG3 respectively).

FIG. 19 illustrates a graph of the resulting activity for the three samples of FIG. 18.

FIG. 20 illustrates a graph of the relative fluorescence of a clinical isolate of S. marcescens digesting a FRET peptide in the absence (HG buffer) or presence of heat gamma autoclaved 1, 2 or 3 times (AHG1, AHG2, and AHG3 respectively).

FIG. 21 illustrates a graph of the resulting activity for the three samples of FIG. 20.

FIG. 22 illustrates a graph of the relative fluorescence of a laboratory strain of Acanthamoeba digesting a FRET peptide in the absence (HG buffer) or presence of heat gamma autoclaved 1, 2 or 3 times (AHG1, AHG2, and AHG3 respectively).

FIG. 23 illustrates a graph of the resulting activity for the three samples of FIG. 22.

FIG. 24 illustrates a graph of the relative fluorescence of a recombinant matrix metalloprotease (MMP 1) digesting a FRET peptide in the absence (HG buffer) or presence of heat gamma autoclaved 1, 2 or 3 times (AHG1, AHG2, and AHG3 respectively).

FIG. 25 illustrates a graph of the resulting activity for the three samples of FIG. 24.

FIG. 26 illustrates a graph of the relative fluorescence of a recombinant matrix metalloprotease (MMP2) digesting a FRET peptide in the absence (HG buffer) or presence of heat gamma autoclaved 1, 2 or 3 times (AHG 1, AHG2, and AHG3 respectively).

FIG. 27 illustrates a graph of the resulting activity for the three samples of FIG. 26.

FIG. 28 illustrates a graph of the relative fluorescence of a recombinant matrix metalloprotease (MMP9) digesting a FRET peptide in the absence (HG buffer) or presence of heat gamma autoclaved 1, 2, or 3 times (AHG 1, AHG2, and AHG3 respectively).

FIG. 29 illustrates a graph of the resulting activity for the three samples illustrated in FIG. 28.

### DETAILED DESCRIPTION OF THE INVENTION

Such protective proteins can also be used to increase cell or tissue viability. The biological activity of the protective proteins enables the protective proteins to be used in the treatment of conditions characterized by cell or tissue damage. The protective proteins can also be used to inhibit protease activity (e.g., elastase, MMP, and bacterial proteases) in cells or tissues and to aid in properly folding proteins that are effective in treating or preventing cell or tissue damage or in maintaining proper folding of such proteins. “Protective proteins” are also known as “Protector Proteins.”

Protective Proteins

The quest to find alternate agents for use in conditions characterized by cell or tissue damage has led to the investigation of the use of “protective proteins” as a tool for the management of these conditions. “Protective proteins” are molecules that have the ability to protect other proteins from damage. In general, these proteins function by minimizing degradation via conformational changes and enzymatic cleavage or digestion, thereby enhancing cell viability in the cells, tissues, and organisms from which they are derived.

As used herein, “protective proteins” are small molecular chaperone proteins isolated from shrimp, mammals (e.g., cows or humans), bacteria, or synthetically produced (e.g., p26, α-A-crystallin, α-B-crystallin, γ-D-crystallin, SicA, high heat stable crystallins) that improve the solubility and/or stability of the proteins. As used herein, the term “protective proteins” also encompasses biologically active fragments, variants, chimeras, and derivatives of protective proteins.

Variants and derivatives of the protective proteins described herein can encompass genetically engineered, synthetic proteins (peptides that have the same or enhanced protective properties of naturally-occurring protective protein). For example, described herein is a peptide of 30 amino acids with enhanced heat stability (SEQ ID NO: 32). The protective proteins can have one or both of the following functions. The first function is protein folding function, and the second function is protease inhibition function. In addition, the protein can be heat stable. Protective proteins including p26 and SicA exhibit the first function, while protective proteins, including α-A-crystallin, γ-D-crystallin and a chimeric recombinant protein, Pepstatin A/Leupeptin/alpha (α-)-A-crystallin (Pep/Leu/α-A-crystallin) exhibit the second function.

As used herein, a p26 protein is a polypeptide comprising or consisting of the amino acid sequence of SEQ ID NO: 1, and amino acid variants, derivatives, analogs, chimeras, and biologically active fragments thereof. A p26 protein is also a polypeptide encoded by the nucleic acid sequence of SEQ ID NO: 2 or nucleic acid variants, derivatives, analogs, chimeras, or fragments thereof p26 is an abundant, low molecular weight, protective chaperone protein of encysted brine shrimp. It is also a member of the small heat shock/alpha crystallin family of proteins.

As used herein, a SicA protein is a polypeptide comprising or consisting of the amino acid sequence of SEQ ID NO: 3, and amino acid variants, derivatives, analogs, chimeras, and biologically active fragments thereof. A SicA protein is also a polypeptide encoded by the nucleic acid sequence of SEQ ID NO: 4 or nucleic acid variants, derivatives, analogs, chimeras, and fragments thereof. SicA is a chaperone protein of the type III excretion system of Salmonella. SicA is another member of the small heat shock protein family.

Thus, the protective proteins help to maintain or increase cell viability upon injury or tissue damage. This results in decreased cell and tissue damage, and faster recovery from injuries or conditions characterized by cell or tissue damage. The protective proteins have one or more of the following activities: inhibiting cell or tissue damage, increasing or maintaining cell or tissue viability, enhancing wound healing, inhibiting protease biological activity, such as matrix metalloprotease protease (MMP) activity, inhibiting the virulence of pathogens such as bacteria (e.g., bacteria that cause tooth decay or gingivitis), and acting as a protease substrate. In one embodiment, the protective protein has one, two, three, four, five, or more than five of the above activities. In another embodiment, the protective proteins have all of the above activities. Subjects can also be treated with the protective proteins. The cell, tissue, or subject to be treated with a protective protein is a vertebrate cell, a mammalian cell, a human cell, tissue (e.g., heart valve tissue, teeth, gum tissue, or bone tissue), a vertebrate, a mammal, or a human. In some embodiments, the protective protein is contacted with (e.g., applied to) normal cells, tissue, or a subject to prevent injuries or infections; in other embodiments, the protective protein is contacted with cells, tissue, or a subject that is suffering from an injury or infection in order to enhance healing, as well as protect from further injury, infection, or damage.

Crystallins

The lens of the mammalian eye is composed of structural fiber cells containing up to 50% protein, making it one of the most unique organs. The water-soluble lens protein crystallin accounts for 90% of the total protein content and 30-35% of the entire mass of the mammalian lens (Hay et al. (1994). Exp. Eye. Res. 58:573-84). Crystallins are the major structural proteins of the vertebrate eye lens. They are a critical component of lens transparency due to their structure, stability and short-range ordering. The generalized function of all crystallins in the maintenance of lens transparency is well documented. Based on the genetic organization, the regulation of their expression pattern and their function in several diseases, crystallins of mammalian lens have been grouped into three main classes; alpha (Δ-), beta (β-) and gamma (γ-) crystallins (Wistow and Piatigorsky (1988). Annu. Rev. Biochem. 57:479-504). It is to be appreciated that additional members of the crystallin family of proteins can still be discovered. Such members will have the same, or closely similar, properties as the crystallin proteins described herein so that one of skill in the art will easily classify the proteins as a member of the crystallin protein family.

Alpha (α-) Crystallins

As used herein, an α-crystallin protein is a polypeptide comprising or consisting of the amino acid sequence of SEQ ID NOs: 5, 7, 17, or 19, and amino acid variants, derivatives, analogs, chimeras, and biologically active fragments thereof. An α-crystallin protein is also a polypeptide encoded by the nucleic acid sequence of SEQ ID NOs: 6, 8, 18, or 20 or nucleic acid variants, derivatives, analogs, chimeras, and fragments thereof. Alpha-crystallins account for up to 35%. of the total protein in vertebrate lenses. Alpha-crystallins are comprised of two subunits (αA and αB) that have structures that are closely related; the subunits can be used together or separately to carry out the methods described herein. The ratio of the cc-crystallin subunits in the hetero-polymer vary depending on the species from which they are isolated and also on the differentiation state of the lens cell (Loutas et al. (1996) Exp. Eye. Res. 62:613-20). The crystallin subunits have significant homology to the heat shock proteins, especially in the C-terminal domain and are therefore considered to be small molecular chaperones.

Alpha (β-) Crystallins

As used herein, a β-crystallin protein is a polypeptide comprising or consisting of the amino acid sequence of SEQ ID NO: 21, and amino acid variants, derivatives, analogs, chimeras, and biologically active fragments thereof. A β-crystallin protein is also a polypeptide encoded by the nucleic acid sequence of SEQ ID NO: 22 or nucleic acid variants derivatives, analogs, chimeras, and fragments thereof. Beta-β-crystallins have been characterized as oligomers with molecular weights ranging up to 200 kDa for octameric forms. Seven forms of β-crystallin polypeptides have been identified and sequenced from bovine lens. They have been divided into two groups: acidic and basic; with four in the acidic group (βA) and three in the basic group (βB). βB 1, with its characteristic long N-terminal extension, has been shown to be required for aggregate assembly. βB2 has been determined to function in maintaining the high solubility to the β-crystallin aggregate. Beta-crystallins share sequence homology with α-crystallin proteins, and therefore, it is reasonable to believe that they possess the biological activity of α-crystallin proteins.

Gamma (γ-) Crystallins

As used herein, a γ-crystallin protein is a polypeptide comprising or consisting of the amino acid sequence of SEQ ID NO: 9, and amino acid variants, derivatives, analogs, chimeras, and biologically active fragments thereof. A γ-crystallin protein is also a polypeptide encoded by the nucleic acid sequence of SEQ ID NO: 10 or nucleic acid variants derivatives, analogs, chimeras, and fragments thereof. Gamma-crystallins exist in monomeric form (20 kDa). Gamma-crystallins are modified in a number of ways during aging and cataract formation. Crystallographic studies using bovine γ-crystallins suggest a capsule like structure that is composed of 8 beta pleated sheets which have a core that is enriched in tyrosine.

Delta (Δ-) Crystallins

As used herein, a Δ-crystallin protein is a polypeptide comprising or consisting of the amino acid sequence of SEQ ID NO: 23, and amino acid variants, derivatives, analogs, chimeras, and biologically active fragments thereof. A α-crystallin protein is also a polypeptide encoded by the nucleic acid sequence of SEQ ID NO: 24 or nucleic acid variants derivatives, analogs, chimeras, and fragments thereof. Delta-crystallins share sequence homology with γ-crystallin proteins, and therefore, it is reasonable to believe that they possess the biological activity of γ-crystallin proteins. Delta-crystallin proteins are also know as arginosuccinate lyase proteins.

High Heat Stable Crystallins

As used herein, a high heat crystallin protein is a polypeptide produced by altering one or more amino acids of a protective protein, or a variant, derivative, analog, chimera, or biologically active fragments thereof described herein, by making conservative amino acid substitutions with amino acids that have a high heat capacity (based on absolute entropies, as described , for example, by Hutchens, et al., “Heat Capacities, Absolute Entropies, and Entropies of Formation of Amino Acids and Related Compounds” in Handbook of Biochemistry, H. A. Sober, ed. (Cleveland, Ohio: Chemical Rubber Co., pp. B60-B61)). As used herein, “high heat” is meant above about 50° C. or in the range of about 50° C. to about 70° C., for example, about 55° C., 60° C., or 65° C. As described herein, heat stable gamma crystallin proteins can be subjected to temperatures in the autoclavable range (e.g., ˜120° C.), for a number of autoclave cycles (e.g., 1, 2, 3, or more than three 25-minutes cycles). In one embodiment the high heat crystallin protein has one or more of the biological activities of a protective protein as described herein. In another embodiment, the high heat crystallin-protein comprises or consists of the amino acid sequence of SEQ ID NO: 15, SEQ ID NO: 32, and amino acid variants, derivatives, analogs, chimeras, and biologically active fragments thereof.

The present invention also pertains to variant nucleic acid molecules (and the use of such nucleic acid molecules) that are not necessarily found in nature but which encode a protective protein as described herein, or another splicing variant of a protective protein or polymorphic variant thereof. For example, DNA molecules that comprise a sequence that is different from the naturally-occurring nucleotide sequence but which, due to the degeneracy of the genetic code, encode a protective protein as described herein are also the subject of this invention. The invention also encompasses nucleotide sequences encoding portions (fragments), or encoding variant polypeptides such as analogs or derivatives (a protein wherein one or more of the atoms of the protein have been replaced with one or more different atoms) of a protective protein. Such variants can be naturally-occurring, such as in the case of allelic variation or single nucleotide polymorphisms, or non-naturally-occurring, such as those induced by various mutagens and mutagenic processes. Intended variations include, but are not limited to, addition, deletion and substitution of one or more nucleotides which can result in conservative or non-conservative amino acid changes, including additions and deletions. Preferably the nucleotide (and/or resultant amino acid) changes are silent or conserved; that is, they do not alter the characteristics or activity of the protective protein.

Polypeptide variants include a substantially homologous polypeptide encoded by the same genetic locus in an organism, i.e., an allelic variant, as well as other splicing variants. Variants also encompass polypeptides derived from other genetic loci in an organism, but having substantial homology to a polypeptide encoded by a protective protein nucleic acid molecule or polymorphic variants thereof. Variants also include polypeptides substantially homologous or identical to these polypeptides-but derived from another organism, i.e., an ortholog, for example, a mammalian ortholog. In one embodiment, the protective protein is a human, mouse, rat, cow, or goat ortholog. Variants also include polypeptides that are substantially homologous or identical to these polypeptides that are produced by chemical synthesis. Variants also include polypeptides that are substantially homologous or identical to these polypeptides that are produced by recombinant methods.

As used herein, two polypeptides (or a region of the polypeptides) are substantially homologous or identical when the amino acid sequences are at least about 45-55%, in certain embodiments at least about 70-75%, and in other embodiments at least about 80-85%, and in other embodiments greater than about 90% or more homologous or identical (e.g., 95%). A substantially homologous amino acid sequence, according to the present invention, will be encoded by a nucleic acid molecule of the invention, or portion thereof, under stringent conditions as more particularly described above, or will be encoded by a nucleic acid molecule hybridizing to a nucleic acid sequence of the invention, portion thereof or polymorphic variant thereof, under stringent conditions as more particularly described thereof.

The invention also encompasses polypeptides having a lower degree of identity but having sufficient similarity so as to perform one or more of the same functions performed by a protective protein encoded by a nucleic acid molecule of the invention. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie, et al. ((1990) Science 247:1306-1310).

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al. (1989) Science 244:1081-1085). The latter procedure introduces single alanine mutations at every residue in the molecule. The resulting mutant molecules are then tested for biological activity in vitro. Sites that are critical for polypeptide activity can also be determined by structural analysis such as crystallization, nuclear magnetic resonance or photoaffinity labeling (Smith et al. (1992) J. Mol. Biol. 224:899-904; and de Vos et al. (1992) Science 255:306-312).

The invention also includes polypeptide fragments of the protective proteins described herein. Fragments can be derived from a protective protein encoded by a nucleic acid described herein. However, the invention also encompasses fragments of the variants of the polypeptides described herein. As used herein, a fragment comprises at least 6 contiguous amino acids. Useful fragments include those that retain one or more of the biological activities of the protective proteins as well as fragments that can be used as an immunogen to generate polypeptide-specific antibodies.

Biologically active fragments (peptides which are, for example, 1-5, 1-10, 1-20, 1-30, 1-40, 1-50, 1-60, 1-70, 1-80, 1-90, or 1-100 or more amino acids in length) can comprise a domain, segment, or motif that has been identified by analysis of the polypeptide sequence using well-known methods, e.g., signal peptides, extracellular domains, one or more transmembrane segments or loops, ligand (protein) binding regions, folding domains, acylation sites, glycosylation sites, or phosphorylation sites. In one embodiment, the domain comprises structural domains of the protective protein, for example, an alpha-helix or a beta sheet. Such domains can be determined using methods known to one skilled in the art. In a particular embodiment the domain comprises the activity that provide the protective protein with its ability to properly fold proteins, for example beta sheets 3 through 5 of the protective protein.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the polypeptide fragment and an additional region fused to the carboxyl terminus of the fragment.

The protective proteins as described herein can be purified from cells that naturally express it, purified from cells that have been altered to express it (recombinant), or synthesized using known protein synthesis methods (For example, as described by Ausubel, F. M. et al. (“Current Protocols in Molecular Biology”, John Wiley & Sons, (1998))). Other methods for purifying protective proteins are described in U.S. patent application Ser. No. 09/848,780 and Ortwerth et al. ((1993) Exp. Eye Res. 56:107-114), the entire teachings of which are incorporated herein by reference. In one embodiment, the protective protein is produced by recombinant DNA techniques (as described, for example, by Ausubel et al., supra). For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques.

Chimeric Protective Proteins

The invention also provides chimeric or fusion proteins comprising protective proteins and/or fragments, variants, analogs, or derivatives thereof. These chimeras comprise two or more protective proteins as described herein, or a protective protein of the invention operatively linked to one or more heterologous proteins or polypeptides having amino acid sequences not substantially homologous to the protective protein. “Operatively linked” indicates that the first protein, e.g., the protective protein, and the second (and/or the third, fourth, fifth, etc.) protein, which can be either a protective or heterologous protein, are fused in-frame. The second protein can be fused to the N-terminus or C-terminus of the polypeptide. In one embodiment the fusion polypeptide does not affect the function of the protective polypeptide per se. In certain host cells (e.g., mammalian host cells), expression and/or secretion of a polypeptide can be increased by using a heterologous signal sequence. Therefore, in another embodiment, the fusion polypeptide contains a heterologous signal sequence at its N-terminus.

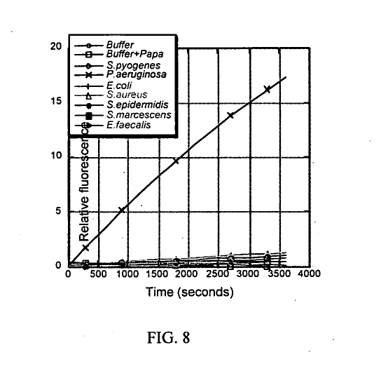

Proteases play an important role in the pathogenesis of conditions characterized by cell and tissue damage, including chronic wounds, keratitis, and gingivitis. Protective proteins, or biologically active fragments thereof, can also be used to generate chimeric proteins that can be used to inhibit cell or tissue damage, enhance cell viability, and/or be used to treat a pathogen-mediated condition by inhibiting proteases. The chimeric protective proteins can comprise, for example, one or more protective proteins and/or one or more protease inhibitors and/or one or more proteins that help maintain proper protein folding. While protective proteins alone can be used to inhibit proteases, in a particular embodiment, the chimeric protein comprises one or more protease inhibitors and a protective protein, for example, alpha(α-)A-crystallin. One example of such a chimeric protein is Pepstatin A/Leupeptin/alpha(α-)A-crystallin (Pep/Leu/α-A-crystallin). In one embodiment, the chimeric protein comprises or consists of the sequence of SEQ ID NO: 14 (FIG. 8K). In another embodiment, the chimeric protein is encoded by the nucleic acid sequence of SEQ ID NO: 16 (FIG. 8L). Such chimeric proteins can be used as a protease inhibitor for treating conditions characterized by cell or tissue damage.

A chimeric or fusion polypeptide can be produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST protein). A nucleic acid molecule encoding a protective protein as described herein can be cloned into such an expression vector such that the fusion moiety is linked in-frame to the polypeptide.

As described above, the chimera protective proteins of the present invention have one or both of the following biological activities: inhibiting cell or tissue damage, increasing or maintain cell or tissue viability, enhancing wound healing, inhibiting protease biological activity, inhibiting the virulence of pathogens, for example, bacteria, and acting as a protease substrate. Methods for measuring these biological activities are known in the art, and are also described herein. Methods for assessing cell or tissue damage and wound healing and for assessing virulence of pathogens include physical examination by a medical professional (e.g., assessment of exudate, erythema, inflammation and induration). Methods for measuring cell viability include assessment using trypan blue or other cell viability stains, commercially available cell viability detection kits and TUNEL kits (e.g., from Promega, Madison, Wis.). Methods for measuring protease biological activity include the use of commercially available kits that detect MMP activity (e.g., from Amersham Biosciences, Piscataway, N.J.) and elastase activity (e.g., Oncolmmunin, Inc., Gaithersburg, Md.).

Compositions

The present invention also pertains to compositions, e.g., pharmaceutical compositions, comprising nucleic acids encoding one or more of the protective proteins described herein or comprising one or more of the protective proteins themselves. For instance, the nucleic acids or protein can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable and animal oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, pastes, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc. Preferred formulations include liquids, for example, solutions that are applied to wounds, or to the eye (e.g., eye wash, eye drops, or contact lens cleaning/disinfecting, rinsing, and/or wetting solutions), ointments, and lotions. Formulations also include compositions suitable for contacting the oral cavity, and in particular, teeth and gums (e.g., mouthwash or toothpaste).

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devices (“gene guns”) and slow release polymeric devices.

The pharmaceutical compositions of this invention can also be administered as part of a combinatorial therapy with other agents, for example, other agents used to treat conditions characterized by cell or tissue damage, including, but not limited to wounds, bums, scar prevention, varicose veins, incisions, for example, made by surgery or other invasive procedures (e.g., insertion of a stent, catheter, medical device, or drug delivery system), meningitis, cosmetic applications, (e.g., bums, irritations, or rashes), ulcers (e.g., antibiotics, first aid cream, ointments, other pharmaceutical agents), inflammation, (e.g., anti-inflammatory agents), damage to the coronary or cardiac systems (e.g., heart valve degeneration or endocarditis), and corneal damage (e.g., medicated or non-medicated eye drops, ointments, or eye washes). The protective proteins of this invention are suitable for use in the therapeutic treatment of transplanted organs or tissue (e.g., transplanted hearts or heart valves). Additionally, the protective proteins of this invention are useful for applications involving skin care (e.g., protecting, reducing, or reversing damage caused by exposure to UV radiation) or other cosmetic application (e.g., stabilizing collagen during or after a cosmetic surgery procedure). The proteins of the present invention can be formulated into toothpastes, powders, or mouth washes to inhibit growth of bacteria associated with tooth decay or gum diseases (e.g., gingivitis). Additionally, the protective proteins are administered intravenously or transdermally.

The composition can be formulated in accordance with the routine procedures as a pharmaceutical composition adapted for administration to human beings. For example, compositions for intravenous administration typically are solutions in sterile isotonic aqueous buffer. Where necessary, the composition can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients can be mixed prior to administration.

For topical application, non-sprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent can be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

In yet another embodiment, the protective protein can be delivered via a controlled release system. Furthermore, a controlled release system can be placed in proximity of the therapeutic target, for example, a wound, wound dressing, an eye, or a contact lens (see, e.g., Goodson, (1984) in Medical Applications of Controlled Release, supra, vol. 2, pp. 115-138). Optionally, the contact lens or wound dressing is the release system as it releases protective proteins at some predetermined rate. For example, a contact lens worn near the cornea of an eye can release protective proteins at a predetermined rate. In a further embodiment, a pump can be used.

In some embodiments, one or more protective proteins are attached or in close contact with a solid support. In further embodiments, the protective protein(s) are absorbed into or onto a solid support. In still more embodiments, the solid support is a material that comes into contact with a wound, infected tissue, and/or inflamed tissue. Examples of suitable solid supports include a wound drainage material, a portion of plastic, a portion of tubing, a suture, a stent, a tissue engineered material (i.e., a material that is made wholly or in part of engineered tissue; e.g., a portion of a heart valve or a portion of material used as a skin substitute or skin replacement), a bioprosthetic material (i.e., a material that includes both tissue and a solid material; e.g., a heart valve that includes cardiac tissue and a support made of plastic, metallic, or ceramic, or a skin replacement material or skin substitute that includes cellular material as well as a supporting material made out of, for example, a polymer or celluloid sheet), a biosynthetic or mechanical implant (e.g., a biosynthetic or mechanical heart valve), or a wound dressing. The wound dressing can be, for example, a bandage, gauze, polymeric material (gels, films, foams), or a sterile pad and the protective protein can be administered (via the protective dressing) to a patient in need thereof. The solid support can comprise material(s) suitable for sterilization. In one embodiment, the protective protein is attached to or absorbed into or onto the solid support and the solid support is then subjected to sterilization. The protective protein can be attached by attaching functional groups to the solid support, followed by coating or impregnating the solid support with the protective protein. In some embodiments, the solid support material comprises one or more functional groups that are used to attach the protective proteins to the solid support.

Methods for attaching proteins to solid supports (for example, wound dressings) are known in the art. For example, proteins with hydrophobic leaving groups can be non-covalently bound to hydrophobic surfaces. Alternatively hydrophilic or hydrophobic proteins can be coupled to surfaces by thiol or primary amine, carboxyl or hydroxyl groups. Free amines can be coupled to carboxyl groups on the protein using, for example, a 10 fold molar excess of either N-ethyl-N′-(3-dimethylaminopropyl)carbodiimide hydrochloride (EDC) or N-cyclohexyl-N′-2-(4′-methyl-morpholinium) ethyl carbodiimide-p-toluene sulphonate (CMC) for 2 hrs at 4° C. in distilled water adjusted to pH 4.5 to stimulate the condensation reaction to form a peptide linkage. SH groups can be reduced with Dithiothreitol (DTT) or Tris[2-carboxymethyl]phosphine hydrochloride] (TCEP) and then coupled to a free amino group on a surface with N-e-Maleimidocaproic acid (EMCA, Griffith et al., FEBS Lett. 134:261-263, 1981).

In one embodiment, the wound dressing can comprise more than one layer, wherein one layer has protective proteins attached to it, while another layer contains material to absorb wound fluid. Optionally, the wound dressing can contain other agents useful for the treatment of conditions characterized by cell or tissue damage. Examples of wound dressing are described, for example, in U.S. Pat. Nos. 6,399,091, 6,399,092, 6,500,539, and 6,471,982, the entire teaching of which are incorporated herein by reference.

In another embodiment, the protective protein is attached to, absorbed into or onto, or in close contact with a contact lens. The lens can be a soft hydrogel lens or a rigid gas permeable lens. The lens can also be a daily wear lens, an extended wear lens, a planned replacement lens, or a disposable lens. In another embodiment the material can be attached to or absorbed into or onto the contact lens and subjected to subsequent steilization. Methods for attaching protective proteins to a solid support (e.g. a contact lens) are described herein.

The protective proteins, or nucleic acids encoding protective proteins are administered to a subject, for example, a mammal in a therapeutically effective amount. The amount of agents which will be therapeutically effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the formulation will also depend on the route of administration, and the seriousness of the symptoms of the conditions to be treated, and should be decided according to the judgment of a practitioner and each patient's circumstances. Effective doses can be extrapolated from dose-response curves derived from in vitro or animal model test systems. In a particular embodiment, the subject is a human.

When the protective proteins are used in solutions for cleaning, disinfecting, rinsing, or storing a contact lens, the lens can be immersed on the solution. Such immersing can comprise soaking and/or rinsing with a steady stream of the solution containing the protective protein. Soaking can optionally comprise shaking or agitating the lens. When the protein is contained in a contact lens rinsing solution, the lens can be inserted or installed into the eye without dilution or rinsing of the solution.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, for use or human administration. The pack or kit can be labeled with information regarding mode of administration, sequence of drug administration (e.g., separately, sequentially or concurrently), or the like. The pack or kit can also include means for reminding the patient to take the therapy. The pack or kit can be a single unit dosage of the combination therapy or it can be a plurality of unit dosages. In particular, the agents can be separated, mixed together in any combination, or present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Therapy

The protective proteins described herein, or nucleic acids encoding the protective proteins can be used in vivo or ex vivo as a treatment or as a prophylactic therapy to prevent cell or tissue damage or a condition characterized by cell or tissue damage. The protective proteins can be used to inhibit cell or tissue damage, increase cell or tissue viability, treat a disease or condition characterized by cell or tissue damage, enhance wound healing, or inhibit protease activity. The cell or tissue damage can be caused by a pathogen (pathogen-induced) or the damage can be non pathogen-induced. Examples of pathogens that can cause, cell or tissue damage include bacteria, viruses, fungi, and parasites. In one embodiment, the cell or tissues to be administered a protective protein is a vertebrate cell, a mammalian cell, for example, a human cell. In another embodiment, the subject to be administered a protective protein is a vertebrate, a mammal, (e.g., a human). Conditions characterized by cell or tissue damage and that can benefit from the therapeutic methods of the present invention include, but is not limited to, wounds (chronic or acute), corneal damage (e.g., microbial keratitis), damage to tissue of the coronary and/or cardiac systems (e.g., heart valves or transplanted heart valves), meningitis, apoptosis, necrosis, bums, scar prevention, varicose veins, incisions, for example, made by surgery or other invasive procedures (e.g., insertion of a stent, catheter, medical device, or drug delivery system), inflammation, and ulcers (e.g., diabetic ulcers, pressure ulcers, venous leg ulcers, corneal ulcers, and peptic ulcers). The inflammation can be caused by or associated with any of the following conditions: diseases involving the gastrointestinal tract and associated tissues (such as appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, coeliac disease, hepatitis, Crohn's disease, enteritis, and Whipple's disease); systemic or local inflammatory diseases and conditions (such as asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, and sarcoidosis); diseases involving the urogenital system and associated tissues (such as septic abortion, epididymitis, vaginitis, prostatitis, and urethritis); diseases involving the respiratory system and associated tissues (such as bronchitis, emphysema, rhinitis, cystic fibrosis, pneumonitis, adult respiratory distress syndrome, alvealitis, bronchiolitis, pharyngitis, pleurisy, and sinusitis); diseases arising from infection by various viruses (such as influenza, respiratory syncytial virus, HIV, hepatitis B virus, hepatitis C virus and herpes), bacteria (such as disseminated bacteremia, Dengue fever), fungi (such as candidiasis) and protozoal and multicellular parasites (such as malaria, filariasis, amebiasis, and hydatid cysts); dermatological diseases and conditions of the skin (such as dermatitis, dermatomyositis, sunburn, urticaria warts, and wheals); diseases involving the cardiovascular system and associated tissues (such as vasulitis, angiitis, endocarditis, arteritis, atherosclerosis, thrombophlebitis, pericarditis, congestive heart failure, myocarditis, myocardial ischemia, periarteritis nodosa, and rheumatic fever); diseases involving the central or peripheral nervous system and associated tissues (such as Alzheimer's disease, meningitis, encephalitis, multiple sclerosis, cerebral infarction, cerebral embolism, Guillame-Barre syndrome, neuritis, neuralgia, spinal cord injury, paralysis, and uveitis); diseases of the bones, joints, muscles and connective tissues (such as the various arthritides and arthralgias, osteomyelitis, fasciitis, Paget's disease, gout, periodontal disease, rheumatoid arthritis, and synovitis); and other autoimmune and inflammatory disorders (such as myasthenia gravis, thryoiditis, systemic lupus erythematosus, Goodpasture's syndrome, Behcets's syndrome, allograft rejection, graft-versus-host disease, Type I diabetes, ankylosing spondylitis, Berger's disease, Type I diabetes, ankylosing spondylitis, Berger's disease, and Retier's syndrome).

In particular, the protective proteins of the present invention can be used to treat infection/inflammation of cells/tissue of the coronary or cardiac system (e.g., heart valves). Also particularly encompassed by the present invention is protection from infection/inflammation of oral cavity tissue, such as gum tissue.

The term “treat” or “treatment” as used herein, refers not only to ameliorating symptoms associated with the disease or condition, but also preventing or delaying the onset of the disease, and also lessening the severity or frequency of symptoms of the disease.

It is understood that a protective protein can inhibit cell or tissue damage or pathogen virulence in varying degrees. For example, the protective protein can decrease cell or tissue damage, protease activity, or pathogen virulence by at least about 10%, 20%, 30%, 40%, 50%, 60%, 70%, 80%, 90%, or 95% relative to an appropriate control, such as a cell or tissue sample or a wound that was not administered protective protein.

It is also understood that a protective protein can increase cell or tissue viability or enhance wound healing to varying degrees. For example, the protective protein can increase cell or tissue viability or enhance wound healing by 10%, 20%, 30%, 40%, 50%, 60%, 70%, 80%, 90%, or 95% relative to an appropriate control, such as a cell or tissue sample or a wound that was not administered protective protein.

The protective protein therapeutic agent (protein or nucleic acid encoding a protective protein) is administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount which will be therapeutically effective in the treatment of a particular individual's disorder or condition will depend on the symptoms and severity of the disease, and can be determined by standard clinical techniques. In addition, in vitro or in vivo assays can optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses can be extrapolated from dose-response curves derived from in vitro or animal model test systems.

In one embodiment, a nucleic acid encoding a protective protein or biologically active fragment thereof can be used, either alone or in a pharmaceutical composition as described above. For example, the nucleic acid, either by itself or included within a vector, can be introduced into cells (either in vitro or in vivo) such that the cells produce protective protein. If necessary, cells that have been transformed with the nucleic acid or a vector comprising the nucleic acid can be introduced (or re-introduced) into an individual affected with the disease or condition. In a preferred embodiment, nucleic acid encoding the protective protein, or a biologically active fragment thereof, can be introduced into an expression vector, such as a viral vector, and the vector can be introduced into appropriate cells in an animal, for example near the site of cell or tissue damage. Other gene transfer systems, including viral and nonviral transfer systems, can be used. Alternatively, nonviral gene transfer methods, such as calcium phosphate coprecipitation, mechanical techniques (e.g., microinjection); membrane fusion-mediated transfer via liposomes; or direct DNA uptake, can also be used.

Wound Healing

Wounds are a type of condition characterized by cell or tissue damage. While acute wounds would benefit from the therapeutic methods as described herein, chronic wounds, which are in particular need for new therapies, would also benefit from the therapeutic methods described herein.

Wound healing is a dynamic pathway that optimally leads to restoration of tissue integrity and function. A chronic wound results when the normal reparative process is interrupted. Chronic wounds can develop from acute injuries as a result of unrecognized persistent infections or inadequate primary treatment. In most cases however, chronic lesions are the end stage of progressive tissue breakdown owing to venous, arterial, or metabolic vascular disease, pressure sores, radiation damage, or tumors. Patients with chronic wounds are typically older people and with an aging population, the incidence of chronic healing failure is likely to increase.

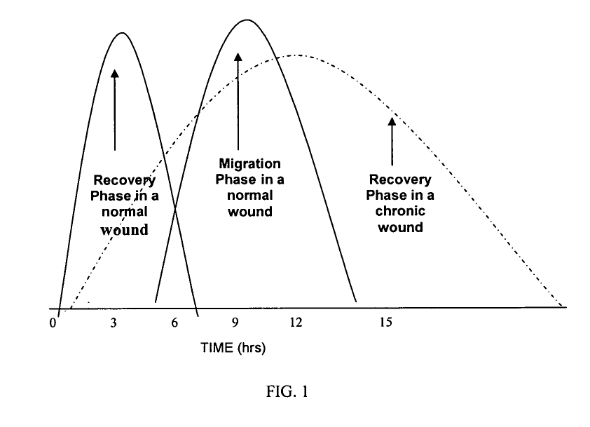

The wound healing process consists of three overlapping phases (FIG. 1). The first phase is an inflammatory phase, which is characterized by homeostasis, platelet aggregation and degranulation. Platelets as the first response, release multiple growth factors to recruit immune cells, epithelial cells, and endothelial cells. The inflammatory phase typically occurs over the first 5 days. The second stage of wound healing is the proliferative phase during which macrophages and granulocytes invade the wound. Infiltrating fibroblasts begin to produce collagen. The principle characteristics of this phase are epithelialization, angiogenesis, granulation tissue formation and collagen production. The proliferative phase typically occurs over 3-14 days. The third phase is the remodeling phase where matrix formation occurs. The fibroblasts, epithelial cells, and endothelial cells continue to produce collagen and collagenase as well as matrix metalloproteases (MMPs) for remodeling. Collagen crosslinking takes place and the wound undergoes contraction. The remodeling phase typically occurs from day 7 to one year.

In chronic wounds, healing does not occur for a variety of reasons, including 1) improper circulation in diabetic ulcers, 2) significant necrosis, such as in bums, and 3) infections. In these chronic wounds, viability or the recovery phase is often the rate-limiting step. The cells are no longer viable and thus initial recovery phase is prolonged by unfavorable wound bed environment.

Wound healing is a complex process involving the interactions of many different cells, matrix components, and biological factors, including growth factors and proteases. Growth factors are polypeptides produced by cells during development and in response to an injury. Growth factors play an important role in providing cells with signaling cues to promote the migration of cells into the surrounding area, affecting cell proliferation, cell migration, and extracellular matrix formation. Growth factors and cytokines regulate the normal process of wound healing in every phase. In the case of chronic wounds there are other mitigating factors that make this healing process more complex. Often the wound bed itself is devoid of extracellular matrix factors such as collagen and laminin that are necessary to coordinate the directed migration of cells. These factors are laid down by fibroblasts, etc., leading to the scaffolding that the tissue can use to start to repair and regenerate. However chronic wounds do not follow the normal process of wound healing, being complicated by the presence of underlying wound pathology. In addition, healing can be complicated by other interfering factors such as infection, tissue hypoxia, advanced age, or an immunocompromised state. Although these factors can inhibit healing by various different mechanisms, in general reduced concentrations of growth factors are found in chronic wounds in addition to high levels of proteases. Both of these situations can contribute to growth factor degradation and unsatisfactory healing (Koveker (2000) Int. J Clin. Pract. 54:590-593; and Stadelmann et al. (1998) Am. J Surg. 176(2A Suppl.):26S-38.).

Chronic Wounds—Growth Factor and MMP Levels

The levels of cytokines present in acute and chronic wounds have been reported in the literature. Pro-inflammatory cytokines such as interleukin-1 and tumor necrosis factor-α were found in high concentrations in chronic leg ulcers (Trengove et al. (2000) Wound Repair Regen. 8:13-25). In many studies, reduced levels of growth factors such as platelet-derived growth factor (PDGF), basic fibroblast growth factor (bFGF), epidermal growth factor (EGF), and transforming growth factor β(TGFβ) have been found in the chronic wound environment when compared to acute wounds.

In contrast, wound fluids from chronic wounds have been found to contain high levels of matrix metalloproteases (MMPs) when compared to acute wounds. The elevated levels of these proteolytic enzymes in the granulation tissue of chronic wounds can contribute to the chronic nature of the wounds. MMPs are present in normal wounds to degrade extracellular matrices, to remove damaged components and to allow cell migration and angiogenesis to occur. However, excessive levels of MMPs can contribute to excess matrix and growth factor degradation in non-healing wounds. (Trengove et al. (1999) Wound Repair Regen. 1999, 7:442-452).

Chronic wounds have been targets for therapeutic wound healing agents. Recent therapeutic approaches have focused on growth factor therapy to address the reduced growth factor levels in chronic wounds or MMP inhibitors to reduce the level of proteases in the chronic wound environment. There have also been some studies on other therapies such as wound dressings and bioengineered matrices (Eming et al. (2002) Cells Tissues Organs 172:05-117).

MMP Inhibitors

Chronic wound fluid contains elevated levels of matrix metalloproteases (MMPs). Matrix metalloproteases (MMPs) are involved in tissue remodeling and disease processes such as arthritis and cancer. In addition to their ability to modify extracellular matrix proteins, MMPs are involved in the regulation of cytokines and cytokine receptors. MMPs catalyze the degradation of collagen and the extracellular matrix. Highly elevated levels of matrix metalloprotease-9 (MMP-9) and elevated levels of MMP-2 in chronic wounds have been reported in the literature, as have elevated levels of MMP-1 and MMP-8 in some chronic wounds (Trengove et al., 1999, supra). Elevated levels of MMPs have been linked to the non-healing environment in chronic wounds, and when chronic wounds do heal, the level of MMPs drops over the course of the healing process. The expression of tissue inhibitors of matrix metalloproteases (TIMPs) has also been studied in chronic wounds and the studies suggest that the balance between MMPs and their inhibitors can be altered in poorly healing wounds (Saarialho-Kere (1998) Arch. Dermatol. Res. 290 Suppl.:S47-S54). TIMPs are a family of homologous proteins that function as endogenous inhibitors of MMPs found in wounds. The levels of active MMP-9 and tissue inhibitor of matrix metalloprotease-I (TIMP-1) in chronic pressure ulcer wound fluid were monitored and it was shown that the average ratio of MMP-9/TIMP- 1 decreases significantly as the wounds healed (Ladwig et al. (2002) Wound Repair and Regen. 10:26-37). Also, in chronic diabetic foot ulcers, decreased concentrations of tissue inhibitor of matrix metalloprotease-2 (TIMP-2) were found (Lobmann et al. (2002) Diabetologia 45:1011-1016).

Thus, agents that decrease MMP to basal levels in wounds or increase TIMP levels or biological activity are useful in the treatment of wounds. Protective proteins as described herein are examples of such agents.

Wound Healing Models

Several animal wound-healing models used to test the efficacy of new therapeutic agents have been developed. Acute wound healing models have been used to test the efficacy of many growth factors. The acute models include full and partial thickness wounds in pigs and guinea pigs and the rabbit ear dermal ulcer model (LeGrand et al. (1995) Wounds 7:78-89; Wang et al. (2003) Arch. Surg. 138:272-279, Mustoe et al. (1991) J Clin. Invest. 87:694-703). Healing-compromised animal models have been developed to try to more closely represent the conditions of a chronic wound. The full thickness wound in a diabetic (db/db) mouse has been used as a model of impaired wound healing (Tsuboi et al. (1992) J Dermatol. 1992, 19:673-675). This model has shown impaired wound healing mainly due to reduced wound contraction although there have been reports that healing in this model can be affected by the type of vehicle and bandaging used.

A chronic infected wound healing model has been used as well. In this model, bacteria inhibit the contraction of the wound (Heggers et al. (1992) J Burn Care Rehabil. 13:512-518). One of the major contributing factors to delayed wound healing is a prolonged inflammatory response in the wound. The presence of bacteria in the wound is known to amplify the local inflammatory response and a prolonged inflammatory response can result in the destruction of tissue by the same processes that normally have restorative functions (Wright et al. (2002) Wound Rep. Regen. 10: 141-151). Other healing-compromised models exist such as the vascular-compromised rabbit ear model. Such animal models can be used to test the efficacy of the protective proteins described herein in treating wounds.

Keratitis

Keratitis, for example, infectious keratitis is another example of a disease that can be treated using the therapeutic proteins and methods of the invention. Infectious keratitis is a complex multifactorial process involving both host and bacterial components. Key predisposing factors for microbial keratitis include extended wear soft contact lens (SCL), ocular surface disease (e.g,. herpetic keratitis, corneal-anesthesia, and bullouskeratopathy) and ocular trauma. SCL have been recognized as the major risk factor for microbial keratitis in otherwise healthy individuals, particularly in the United States (Dart et al. (1991) Lancet 338:650-3). The risk of corneal infection among contact-lens wearers has been estimated to be 80 times greater than that of healthy non-wearers.

P. aeruginosa and Its Role in Microbial Keratitis

The high prevalence of P. aeruginosa infection has been associated with the rising popularity of contact lenses. P. aeruginosa has also been the most frequent ocular isolate due to its limited nutrient requirement for growth and due to its remarkable ability to adhere to the contact lens. P. aeruginosa mediated liquefactive destruction of the cornea results from the release of a multitude of virulence factors including, but not limited to, alkaline protease (aeruginolysin), elastase (pseudolysin), staphylolysin, exotoxin A, phospholipase C and protease IV (Lomholt et al. (2001) Infect. Immun. 69:6284-95).

Of the virulence factors, the role that elastases (a zinc metalloproteinase) and alkaline proteases play in inducing necrosis of the corneal tissues has received much attention (Howe and Iglewski (1984) Infect. Immun. 43:1058-63; Kernacki et al. (1995) Invest. Ophthaimol. Vis. Sci. 36:1371-8; Kreger and Gray (1978) Infect. Immun. 19:630-48; and Twining et al. (1986) Curr. Eye Res. 5:503-10). Therefore, administration of agents, for example protective proteins, that decrease or prevent elastase activity would be a useful method for treating corneal damage.

The evasion of the host defense system also plays a vital role in the ability of a pathogen to successfully colonize, proliferate and establish infection. P. aeruginosa has been shown to counter the host immune system either by the production of large quantities of extracellular products (proteases, toxins and lipases) or by the formation of biofilms, which provides a barrier against the immune components.

Pseudomonas proteinases, alkaline proteases, and elastases have been shown to interfere with the host immune system via the degradation of a large number of both humoral and cellular components including, tumor necrosis factor-α, interleukin 2, components of the complement cascade, immunoglobulin G (IgG), and IgA (Kharazmi (1991) Immunol. Lett. 30:201-5; and Parmely et al. (1990) Infect. Immun. 58:3009-14).

The importance of bacterial exoproducts in microbial keratitis has been further substantiated by passive immunization studies using purified Pseudomonas protease preparations. Immunization with purified proteases prior to corneal challenge with live. P. aeruginosa was found to significantly reduce corneal damage when compared to the unimmunized control. These results were in agreement with previous immunization studies performed by other investigators and thus bolster the key role of proteases in infectious keratitis (Tanaka et al. (1977) Nippon Ganka Gakkai Zasshi 81:1252-9; and Ueda et al. (1982) Nippon Juigaku Zasshi 44:289-300).

Therefore, there is a need for therapeutics that can be delivered to the eye, and to objects or solutions that contact the eye (e.g., contact lenses, contact lens cleaning solutions, ointments, and eyedrops) to inhibit the virulence of Pseudomonas. Protective proteins as described herein can be used for such a purpose. The protective proteins can also be administer to individuals infected with a pathogen, to protect the individual against cell or tissue damage caused by the pathogen.

Role of Host Derived Pathogenic Factors in Microbial Keratitis

The exoenzymes secreted by the bacteria play a major role in the initial pathogenic events leading to keratitis, however, these factors are replaced within 24-48 hours by destructive enzymes secreted by activated polymorphonuclear leukocytes cells (PMNs) (Hazlett et al. (1990) Curr. Eye. Res. 9:129-38) and the resident host cells (corneal, epithelial, and stromal) (Lausch et al. (1996) J Interferon Cytokine Res. 16:35-40; Steuhl et al. (1987) Invest. Ophthalmol. Vis. Sci. 28:1559-68; Steuhl et al. (1989) Fortschr. Ophthalmol. 86:283-6; and Xue, et al. (2002) Clin. Experiment. Ophthalmol. 30:196-9). The activation of PMNs is mediated via the pro-inflammatory cytokines (interleukin-6 (IL-6), IL-1b, IL-8 and tumor necrosis factor alpha (TNF-(α)) (Thakur et al. (2002) Infect. Immun. 70:2187-97). Although PMNs play a major role in bacterial clearance, their prolonged presence has also deleterious implications on the host. For example, PMNs activate matrix metalloproteinases (MMPs) which cause irreversible corneal damage (Miyajima et al. (2001) Microb. Pathog. 31:271-81; and Okada and Nakanishi (1989) FEBS Lett. 249:353-6). Increased levels of the active form of MMPs occur during microbial keratitis.

Therefore, there is a need for therapeutics that can be delivered to the eye, and to objects and solutions that contact the eye to inhibit MMP levels and/or activity. Protective proteins as described herein can be used for such a purpose.

Cationic Antimicrobial Peptides: An Alternative Approach for the Management of Bacterial Keratitis

Antimicrobial host defense peptides have been well recognized as effector molecules of the innate immune system and are widely distributed among species. These peptides possess cationic properties that facilitate interactions with the negatively charged phospholipids of the bacterial membrane. In humans, three types of cationic antimicrobial peptides (CAMPs) have been identified: defensins, cathelicidins, and thrombocidins.

Defensins are the largest class of anti-microbial peptides secreted by mammalian cells (neutrophils, intestinal Paneth cells, and barrier epithelial cells). They counter microbial, fungal, as well as viral infection and thus serve as a first line of defense against the invading pathogens. In addition to the role of defensins in host defense, they are thought to play a role in inflammatory disorders, wound repair, and the promotion of adaptive immune responses. These cationic proteins/polypeptides are arginine rich and possess six cysteine residues that form three intramolecular disulfide bridges, a hallmark feature of defensins. Based on spacing of the cysteine residues and alignment of disulphide bridges, three groups of mammalian defensins have been identified to date, which include (i) a (human neutrophil peptide, HNP), (ii) b (human beta-defensins, hBD-2) and (iii) q (circular, or ‘theta” minidefensin) subfamily. The role of defensins as a potential therapeutic agent has been substantiated by recent findings of Tang et al., who have shown direct involvement of α-defensins in slowing the progression of HIV infection in infected individuals. Defensins also play an important role in fighting E. coli infections.

Cathelicidins are a class of anti-microbial peptides encoded exclusively by mammals. They are bipartite molecules with an N-terminal cathelin domain and a C-terminal antimicrobial domain (LL-37). In humans, cathelicidin is encoded by the hCAP-18 gene. The serine protease, proteinase 3, facilitates the cleavage of the active peptide LL-37 (antimicrobial domain of hCAP-18), which then exerts antibacterial activity against both Gram-positive and Gram-negative bacteria. More recent studies have shown that LL-37 can act in synergy with the well-characterized antimicrobial peptide, a-defensin, to clear bacterial infections. In addition to host defense, this class of gene-encoded antibiotics are involved in additional functions which include chemotactic activity, mitogenesis, and angiogenesis. Due to the involvement of these peptides in diverse functions, they are also termed as “multifunctional effector molecules.”

Thrombocidins are truncated versions (carboxy terminal deletions) of the CXC chemokines which are released upon the activation of the blood platelets, hence the name thromobocidins. They have been previously demonstrated to clear Streptococcal, Staphylococcal and Candida infections in in vitro experimental models.

Antimicrobial peptides kill by permeabilizing the membrane of microbial organisms. Two to four molecules of defensin aggregate and form voltage dependent channels in the lipid bilayer, resulting in the permeabilization of both the inner and outer membranes of the microorganism (Lehrer et al. (1989) J Clin. Invest. 84:553-61). The amphiphilic nature of the defensins facilitates the insertion of the hydrophobic residues into the lipid bilayer by electrostatic attraction, while the polar residues project into and above the membrane.

Bacterial Resistance to Antimicrobial Peptides Resistance to CAMPs is one of the key virulence factors of a successful pathogen. Pathogenic microorganisms have evolved several mechanisms by which they counteract CAMPs including covalent modifications of anionic molecules to reduce the negative charge of the bacterial cell envelope, efflux of CAMPs via the proton-motive-force dependent efflux pumps; alteration of the membrane fluidity, and degradation of CAMPs by proteolytic cleavage.

The degradation of CAMPs by proteinases of pathogenic bacteria has been characterized. Pathogenic bacteria secrete proteinases such as elastase, cysteine proteases, and alkaline proteinases, which modify host responses such as kallikreins, coagulation factors, complement, cytokines, and antiproteinases (Travis et al. (1995) Trends Microbiol. 3:405-7). The fact that proteinases play a major role in microbial virulence has been further substantiated by recent in vitro findings that proteinases secreted by Pseudomonas aeruginosa, Enterococcusfaecalis, Proteus mirabilis, and Streptococcus pyogenes degrade antibacterial peptides, in particular LL-37 (a well studied member of the cathelicidin class of CAMPs) (Schmidtchen et al. (2002) Mol. Microbiol. 46:157-68). Therefore, the protective proteins of the present invention can be used to decrease or prevent CAMP degradation, thereby boosting the innate host defense system, and to protect the body against cell or tissue damage caused by pathogens and/or compounds secreted by pathogens.

Methods for detecting protein degradation, (e.g., gel electrophoretic techniques, protein purification techniques) are known to one skilled in the art and can be used to detect proteolysis of CAMPs after treatment with a protective protein.

In one embodiment, the pathogen causes cell or tissue damage through the following diseases or conditions:

bacterial diseases, for example, staphylococcus infections (caused, for example, by Staphylococcus aureus, Staphylococcus epidermis, or Staphylococcus saprophyticus), streptococcus infections (caused, for example, by Streptococcus pyogenes, Streptococcus pneumoniae, or Streptococcus agalactiae), enterococcus infections (caused, for example, by Enterococcusfaecalis, or Enterococcusfaecium), diphtheria (caused, for example, by Corynebacterium diptheriae), anthrax (caused, for example, by Bacillus anthracis), listeriosis (caused, for example, by Listeria monocytogenes), gangrene (caused, for example, by Clostridium perfringens), tetanus (caused, for example, by Clostridium tetanus), botulism (caused, for example, by Clostridium botulinum), toxic enterocolitis (caused, for example, by Clostridium difficile), bacterial meningitis (caused, for example, by Neisseria meningitidis), bacteremia (caused, for example, by Neisseria gonorrhoeae), E. coli infections, including urinary tract infections and intestinal infections, shigellosis (caused, for example, by Shigella species), salmonellosis (caused, for example, by Salmonella species), yersinia infections (caused, for example, by Yersinia pestis, Yersinia pseudotuberculosis, or Yersinia enterocolitica), cholera (caused, for example, by Vibrio cholerae), campylobacteriosis (caused, for example, by Campylobacter jejuni or Campylobacter fetus), gastritis (caused, for example, by Helicobacter pylori), pseudomonas infections (caused, for example, by Pseudomonas aeruginosa or Pseudomonas mallei), haemophilus influenzae type B (HIB) meningitis, HIB acute epiglottitis, or HIB cellulitis (caused, for example, by Haemophilus influenzae), pertussis (caused, for example, by Bordetella pertussis), mycoplasma pneumonia (caused, for example, by Mycoplasma pneumoniae), nongonococcal urethritis (caused, for example, by Ureaplasma urealyticum), legionellosis (caused, for example, by Legionella pneumophila), syphillis (caused, for example, by Treponema pallidum), leptospirosis (caused, for example, by Leptospira interrogans), Lyme borreliosis (caused, for example, by Borrelia burgdorferi), tuberculosis (caused, for example, by Mycobacterium tuberculosis), leprosy (caused, for example, by Mycobacterium leprae), actinomycosis (caused, for example, by Actinomyces species), nocardiosis (caused, for example, by Nocardia species), chlamydia (caused, for example, by Chlamydia psittaci, Chlamydia trachomatis, or Chlamydia pneumoniae), Rickettsial diseases, including spotted fever (caused, for example, by Rickettsia ricketsii) and Rickettsialpox (caused, for example, by Rickettsia akari), typhus (caused, for example, by Rickettsia prowazekii), brucellosis (caused, for example, by Brucella abortus, Brucella melitensis, or Brucella suis), and tularemia (caused, for example, by Francisella tularensis);

viral diseases or conditions caused by infection with any of the following viruses: Influenza Virus (Influenza), Parainfluenza, Respiratory Syncytial Virus (Respiratory Syncytial Virus Infection), Adenoviruses, Rhinoviruses, Coronaviruses, Reoviruses, Mumps Virus (Mumps), Measles Virus (Measles), Rubella Virus (Rubella Infection), Parvovirus B19, Poxviruses, including Variola Virus (Smallpox), Vaccinia Virus, Molluscum Contagiosum Virus, Orf Virus, Pseudocowpox Virus, Cowpox virus, Mousepox Virus, Monkeypox Virus, and Buffalopox Virus, Enteroviruses, including Poliovirus (Polio), Coxsackievirus, Echovirus, Hepatitis Viruses, including Hepatitis A, Hepatitis B, Hepatitis C, Hepatitis D, and Hepatitis E, Herpesviruses, including Herpes Simplex Virus, Cytomegalovirus, Varicella-zoster Virus (Chickenpox), Herpes Zoster (Shingles), Epstein-Barr Virus (Infectious Mononucleosis), and Human Herpesvirus Type 6 (Roseola), Rotaviruses (intestinal disorders), Norwalk Viruses (intestinal disorders), Togaviruses, Flaviviruses, Bunyaviruses, Reoviruses, Arenaviruses, Arboviruses (Western Equine Encephalitis, Eastern Equine Encephalitis, St. Louis Encehphalitis, California Virus, Yellow Fever, Dengue, Japanese B Encephalitis, Powassan Virus, and Colorado Tic Fever), Filioviruses (Ebola Virus and Marburg Virus), Hantaviruses (Hantavirus Hemorrhagic Fever), Vesicular Stomatitis Viruses, Rabies Viruses (Rabies) Retroviruses, including HIV Viruses, and Papovaviruses, including Papillomaviruses, and Polyomaviruses;

fungal diseases, for example, dermatophytoses (caused, for example, by species or the genera Microsporum, Trichophyton, or Epidermpphyton), candidiasis (caused, for example, by Candida albicans, Candida krusei, Candida parapsilosis, Candida tropicalis, or Candida guilliermondii), aspergilliosis (caused, for example, by Aspergillus species), zygomycosis (caused, for example, by Absidia, Phizopus, and Mucor zygomycetes), blastomycosis (caused, for example, by Blastomyces dermatitidis), cryptococcosis (caused, for example, by Cryptococcus neoformans), histoplasmosis (caused, for example, by Histoplasma capsulatum), coccidioidomycosis (caused, for example, by Coccidioides immitis), sporotrichosis (caused, for example, by Sporothrix schenckii) and pneumocystosis (caused, for example, by Pneumocystis carini); and

parasitic diseases, for example, helminthic diseases, such as enterobiasis (caused, for example, by Enterobius vermicularis), trichuriasis (caused, for example, by Trichuris trichuria), ascariasis (caused, for example, by Ascaris lumbricoides), hookworm disease (caused, for example, by Necator americanus and Necator duodenale), cryptosporidiosis (caused, for example, by Cryptosporidium parvum), amebiasis (caused, for example, by Entamoeba histolytica), amebic meningoencephalitis (caused, for example, by amebas of the Naegleria and Ancanthamoeba genera), toxoplasmosis (caused, for example, by Toxplasma gondii), malaria (caused, for example, by Plasmodium vivax, Plasmodium ovale, Plasmodium malariae, or Plasmodium falciparum), trichomoniasis (caused, for example, by Trichomonas vaginalis), giardiasis (caused, for example, by Giardia lamblia), leishmaniasis (caused, for example, by Leishmania tropica, Leishmania mexicana, Leishmania braziliensis, Leishmania donovani), African trypanosomiasis (caused, for example, by flies of the genus Glossina), and American trypanosomiasis (caused, for example, by Trypanosoma cruzi).

The cell or tissue damaged caused by the pathogen can be caused by a substance or an agent released from the pathogen, also referred to herein as a factor, such as a virulence factor. Examples of such substances include proteins or toxins. In one embodiment the pathogen is Pseudomonas aeruginosa and/or one or more of the species described herein.

Heart Valves

Heart valve replacement refers to the procedure used to replace one of the heart valves rather than repairing it. The damaged heart valve is removed and a prosthetic valve is sewn into the remaining tissue from the natural valve. Worldwide, 95% of valve replacements are performed for mitral or aortic valves. Generally, two types of prosthetic heart valves are used for valve replacement: mechanical or tissue. Mechanical heart valves are produced from man-made materials and are very durable. These valves can last the lifetime of a valve recipient although they are prone to develop blood clots so the patient must remain on anticoagulation medication for their lifetime. Tissue valves are composed of natural tissue and typically do not require lifelong anticoagulation medication. However, tissue valves do not last as long as mechanical valves.

Bioprosthetic (tissue) heart valves have been used for over 30 years and more than 50% of the bioprosthetic valves fail within 15 years due to structural deterioration. The ability to stabilize animal heart valves with glutaraldehyde has been of significant value in the use of bioprosthetic heart valves. Over the last 30 years, the tissue derived heart valves have been improved by design changes and small changes in the processing of the tissue. However, the durability of tissue heart valves has not been significantly improved and the valves have a tendency to calcify. It has been shown that glutaraldehyde-fixed bioprosthetic heart valves are not metabolically inert and not entirely resistant to enzymatic degradation over time. Nimni, M. E., Glutaraldehyde Fixation Revisted, J. Long Term Eff. Med. Implants, 2001, 11(3-4): 151-61.

Tissue deterioration, manifested as calcification and mechanical damage to the extracellular matrix, limits the durability of bioprosthetic heart valves. There are several biochemical pathways which lead to tissue degeneration of heart valves and typically failure is divided into two categories: calcific and non-calcific degeneration. Calcification of bioprosthetic heart valves occurs when crystalline hydroxyapatite is deposited in the valve, and ultimately leads to a lack of flexibility and mechanical integrity of the valve. The calcification of the valve often leads to stenosis and tearing of the tissue, which can cause regurgitation and ultimate failure of the valve. Non-calcific failure results in the loss of the integrity of the valve in the absence of calcium deposits. It has been suggested that calcific and non-calcific degradation are interrelated as hydroxyapatite crystals often deposit near damaged collagen or elastin. It has been shown that calcification occurs both in the cusp and the aortic wall portions of prosthetic heart valves. Elastin has also been shown to have a significant role in the calcification of both the cusp and aortic wall portion of tissue valves. (Singla, A. and Lee, C. H., “Effect of Elastin on the Calcification Rate of Collagen-Elastin Matrix Systems”, J. Biomed. Mater. Res., 2002 60(3): 368-374; Bailey, M. T., Pillarisetti, S., Xiao, H., and Vyavahare, N. R., “Role of Elastin in Pathologic Calcification of Xenograft Heart Valves”, J. Biomed. Mater. Res., 2003, 66A(1): 93-102).

Further studies have shown that the inhibition of MMP activity can play a role in reducing elastin-orientated calcification. (Vyavahare, N., Jones, P., Tallapragada, S., and Levy, R., “Inhibition of Matrix Metalloproteinase Activity Attenuates Tenascin-C Production and Calcification of Implanted Purified Elastin in Rats”, Am. J. Path., 2000, 157: 885-893.) The proteolytic activity of MMPs, both intrinsic to the tissue valve and host-derived, has been implicated in heart valve failure. It has been shown that active soluble and insoluble proteases exist within glutaraldehyde-fixed bioprosthetic heart valves. Proteolytic enzymes capable of degrading collagen and elastin were found in unimplanted glutaraldehyde-fixed bioprosthetic heart valves. There is also evidence that the extracellular matrix components of these valves are susceptible to tissue enzymes capable of degrading glutaraldehyde-fixed collagen and particularly elastin. (Simionescu, A., Simionescu, D., and Deac, R., “Biochemical Pathways of Tissue Degeneration in Bioprosthetic Cardiac Valves; The role of matrix metalloproteinases”, ASAIO J., 1996, 42(5): M561-567; Bracher, M., Simionescu, D., Simionescu, A., Davies, N., Human, P., and Zilla, P., “Matrix Metalloproteinases and Tissue Valve Degeneration”, J. Long Term Eff. Med. Implants, 2001, 11(3-4): 221-230; Simionescu, D., Lovekamp, J., and Vyavahare, N., “Extracellular matrix degrading enzymes are active in porcine stentless aortic bioprosthetic heart valves”, J. Biomed. Mater. Res., 2003, 66A: 755-763).

Additionally, tissue inflammation can play a role in heart valve degeneration. Several studies found that immunological reactions do play a role in valve degeneration. The immunogenicity of the xenograft is not fully masked and can depend on the level of glutaraldehyde crosslinking. (Human, P. and Zilla, P., “Inflammatory and Immune Processes: The Neglected Villain of Bioprosthetic Degeneration?”, J. Long Term Eff. Med. Implants, 2001, 11(3-4): 199-220.; Wilhelmi, M. H., Mertsching, H., Wilhelmi, M., Leyh, R., and Haverich, A., “Role in Inflammation in Allogenic and Xenogeneic Heart Valve Degeneration: Immunohistochemical Evaluation of Inflammatory Endothelial Cell Activation”, J. Heart Valve Dis., 2003 12(4): 520-526).

A further factor in tissue valve failure is mechanical stress. Studies have isolated mechanical factors alone and studied their role in altering biomechanical properties of the valve and their contribution to the fatigue damage process. These studies have indicated that there is more than the loss of stiffness of the collagen fibers that contributes to mechanical degradation. Collagen fiber disruption and degradation to the extracellular matrix can also lead to mechanical failure, relating mechanical damage to enzyme degradation. (Sacks, M. S., “The Biomechanical Effects of Fatigue on the Porcine Bioprosthetic Heart Valve”, J. Long Term Eff. Med. Implants, 2001, 11(3-4): 231-247; Sacks, M. S., and Schoen, F. J., “Collagen Fiber Disruption Occurs Independent of Calcification in Clinically Explanted Bioprosthetic Heart Valves”, J. Biomed. Mater. Res., 2002, 62(3): 359-371)

Therefore a need exists for methods and therapeutics that can alleviate one or more of these factors that contribute to the degradation of tissue valves, thereby inhibiting or slowing the degradation of tissue heart valves. Protective proteins, as described herein, are useful for such a purpose. The protective proteins can be administered to individuals to prevent, slow, halt, and/or reverse the degradation of heart valves (e.g., an original heart valve or an implanted tissue valve). For example, a therapeutic agent comprising one or more protective proteins can be administered to a mammal (e.g., a human) to prevent slow, halt, and/or reverse the degradation of heart valves.

In some embodiments, one or more protective proteins are used to treat endocarditis (i.e., inflammation of the endocardium lining the interior of the heart) and/or artherosclerosis. In further embodiments, one or more catheters and/or stents comprising one or more protective proteins are used to prevent or reduce inflamation and damage to the linings on the walls of at least some portion of cardiac and/or pulmonary systems. In further embodiments, the protective proteins of this invention are used in skin care and plastic surgery applications of the protective proteins (i.e. protection of UV rays and damage to skin due to harsh conditions). In addition, the protective proteins can also be used to prevent inflammation associated with meningitis and prevent prion formation.

In one embodiment, protective proteins are administered as a pharmaceutical preparation as described herein to treat original or implanted heart valves. In another embodiment, protective proteins are coated, adsorbed into or onto, or attached to or in close contact with materials used in heart valve replacement prior to implantation. For example, protective proteins can be coated, adsorbed into or onto, attached to or in close contact with bioprosthetic heart valves. The protective protein can be added to the bioprosthetic heart valve before or after treatment with glutaraldehyde or other treatments. The protective protein can also be coated, adsorbed into or onto, attached to or in close contact with the cuff of bioprosthetic and mechanical heart valves.

In one embodiment, a protective protein of the invention includes the amino acid sequence MGKITLYEDR GFQGRHYECS (SEQ ID NO: 27). In a further embodiment, a protective protein of the invention includes the amino acid sequence MGKITLYEDR GFQGRHYECS SFDKDGNGYI SAAELRHVMT NLGEKLTDEE VDEMIREADI DGDGQVNYEE FVQMMTAK (Gamodulin; SEQ ID NO: 28). Gamodulin, a chimeric protein, comprises the amino terminal domain of gamma-crystallin and the calcium-binding domain (EF hand) of calmodulin. Gamodulin can act as a protease inhibitor. Gamodulin can also bind to or sequester calcium.

Dental Disease

P. gingivalis is the organism most strongly associated with, and is one of the major etiological agents of, adult periodontal disease. Several virulence facts have been identified, including proteinases and adhesion factors. The majority of the P. gingivalis proteinases are collagenases and trypsin-like enzymes, with the majority of the trypsin-like enzymes being cysteine proteases with cleavage specificity after lysine or arginine residues. These cysteine proteases are commonly referred to as “gingipains.” The arginine specific proteases are referred to as Gingipain R and the lysine specific proteases are referred to as Gingipain K. Lysine specific gingipain is encoded by a single gene, kgp, while the arginine specific gingipain is encoded by two related genes, rgpA and rgpB.

In some embodiments, the protective proteins are incorporated into or on a dental product. For example, the protective proteins of the invention can be included in a fluid for rinsing the mouth of a mammal (e.g., a human) or in toothpaste. In other embodiments, the protective proteins are included in or on a dental article, such as a tooth brush, a cotton ball or other absorbent article typically used during or after dental procedures, dental floss, tooth pics, a restorative material (e.g., a dental composite, amalgam, or denture), a surgical suture or stitching material, or a polymeric material useful for protecting or cover stitches and sores in the mouth. In further embodiments, the protective proteins are rinsed, injected, film sprayed, or aerosolized onto tissue in an oral cavity (e.g., gum tissue or teeth) of a mammal (e.g., a human) or onto articles of dental application.

The invention will be further described by the following non-limiting examples. The teachings of all publications cited herein are incorporated herein by reference in their entirety.

### EXAMPLE 1

### Cloning, Over-Expression, and Purification of p26, α-A-crystallin, γ-D-crystallin, SicA and the Chimerical Protein, Pepstatin A/Leupeptin/Alpha(α-)A-crystallin

Oligonucleotide primers were designed to incorporate an NdeI restriction site at the 5′ end and an XbaI site at the 3′ end of the gene during the amplification of alpha(α-)A-crystallin, gamma (γ-)D-crystallin from human cDNA clones (ResGen Genestorm Clone, Invitrogen, Carlsbad, Calif.). To generate a robust protease inhibitor a recombinant chimeric protein of PepstatinA (aspartic protease inhibitor), Leupeptin (inhibitor of serine proteases and some cysteine protease) and alpha(α-)A-crystallin (elastase inhibitor) (Pep/Leu/α-A-crystallin) was constructed. The chimeric protein was designed so as to have a broad range of specificity for inhibiting proteases. This chimera was generated by a two-step cloning procedure of the open reading frames (ORFs) into the expression vector.

Cloning and expression of the recombinant crystallin proteins were performed using standard techniques. Briefly, the PCR amplified fragments were cloned into the NdeI/XbaI site of either the expression vector pET28a (to generate poly-histidine tagged proteins) or pET24a (to generate untagged proteins) vectors (Novagen, San Diego, Calif.). The presence of the insert in the putative clones was confirmed by colony PCR and sequencing of the constructs. The overnight cultures of the transformants were used to inoculate 1 liter of fresh Luria Bertani (LB) broth supplemented with kanamycin (30 μg/mL). At an optical density A₆₀₀ of 0.4, the cells were induced for 2 hours to express the recombinant proteins by the addition of 1 mM isopropyl-β-D-thiogalactopyranoside (IPTG, Sigma, St. Louis, Mo.) and purified from E. Coli cells using standard size exclusion, ion exchange, and/or affinity chromatography resins. The yields were typically 5-20 mg/L.

Cloning and expression of the recombinant p26 and SicA proteins were performed using standard techniques. Briefly, the proteins were subcloned into expression plasmids using standard techniques. The plasmids were electroporated into E. coli cells, which were plated and incubated overnight at 37° C. Colonies containing the expression plasmid were then cultured, and protein induction was induced using IPTG. The proteins were then purified using standard protein purification techniques known to one of skill in the art.

### EXAMPLE 2

### “Foldase” Assay

As described herein, protective proteins can function as general-purpose folders for any molecule that has formed unstable protein intermediates. A “foldase” assay to quantify the folding activities of these proteins has been developed. A labile, heat-sensitive enzyme is heated with the protective protein and activity is then measured and compared to a control.

Nde I is a restriction endonuclease which can linearize the plasmid vector pET28a (Novagen, San Diego, Calif.) by cleaving its unique Nde I site. After 60 minutes of pre-incubation at 40° C., the activity of Nde I is significantly diminished, and the enzyme can no longer filly cleave pET28a. Here it is demonstrated that p26 is capable of enhancing and prolonging the activity of Nde I under these conditions. Three reaction tubes were prepared as described in Table 1.

TABLE 1

| Reaction # | Enzyme | Protein added | Pretreatment |
|---|---|---|---|
| 1 | Nde I (20 units) | None (buffer control) | 40° C. for 1 hour |
| 2 | Nde I (20 units) | 5 μg p26 | 40° C. for 1 hour |
| 3 | Nde I (20 units) | 5 μg BSA | 40° C. for 1 hour |

Each of the three mixtures was incubated at 40° C. for 60 minutes. Immediately thereafter, 0.9 μg of uncut pET-28a DNA was added to each tube, and the mixtures were then incubated at 37° C. for an additional 60 minutes. Immediately after the second incubation, the digests was analyzed by agarose gel electrophoresis. An ethidium stained gel was produced. After a one hour pretreatment in buffer alone, Nde I was partially inactivated, and could no longer fully cleave pET-28a (lane 4). In the presence of 5 μg of p26 protein, Nde I remained fully active, and completely cleaved pET-28a (lane 5). For comparison, the enzyme was heated in the presence of bovine serum albumin (BSA), a protein commonly used to stabilize proteins. An equivalent amount of BSA (5 μg) did not protect the enzyme any better than the buffer control (lane 6). These results show that p26 helps to maintain proteins in proper conformation, thereby enabling their function.

### EXAMPLE 3

### Protective Proteins Enhance Wound Healing

Wound healing is a multi-phase process involving two important and distinct activities: cell migration and cell recovery. Most of the effort in this field has been focused on the roles of growth factors (e.g., PDGF) and extracellular components (e.g., collagen) in cell migration. In contrast, the approach described herein focuses on proteins that actively promote cell viability by minimizing denaturation due to unfolding or other conformational changes in protein structure and/or due to enzymatic cleavage. Both mechanisms of denaturation are significant problems in chronic wounds.

Based on the understanding of the ability of the protective proteins to stabilize proteins in vitro, it is reasonable to believe that the protective proteins promote the healing of chronic wounds through the enhancement of cell viability. This was tested in an infected partial thickness porcine model of chronic wounds. The pig is a widely used model for the evaluation of wound infections and wound healing because of the similarity of pig integument to that of the human skin. The purpose of this animal study was to determine if the protective proteins (p26 and Pep/Leu/α-A-crystallin) were able to accelerate the healing of chronic wounds infected with 10⁵ CFU of Pseudomonas aeruginosa. It has previously been established in this porcine model that the P. aeruginosa infected wounds did not heal within 5 days.

To perform the porcine wound infection assay, a dermatome was used to produce fourteen uniform partial thickness wounds on the back of a pig (2.5 cm×2.5 cm×0.8 mm) under sterile conditions. The wounds were infected with 10⁵ CFU of Pseudomonas aeruginosa in sterile PBS. A negative control (sterile PBS) was added to one pair of wounds. The inoculate was spread evenly over the entire wound surface. Protective proteins were added once a day for five days at a concentration of 10-30 μg in duplicate wounds. Wounds were covered with bioclusive film and spaced 3 cm apart to avoid cross-contamination. Photographs were taken each day during a dressing change. The wounds were also scored based on visual observations of exudate, erythma, inflammation, and induration. Edema and distance to closure were also measured.

Results demonstrated that p26 and Pep/Leu/α-A-crystallin accelerated the healing of the wounds in 4 days while slower healing was observed with untreated (PBS negative control) wounds (all performed in duplicate). The wounds treated with protective proteins appeared to have achieved a barrier to protect the wound from outside elements. The control wounds remained open and red. The results of this study show that protective proteins accelerated healing of the wounds when compared to untreated control wounds.

### EXAMPLE 4

### Inhibition of Enzyme Activity Using Protective Proteins

An array of specific enzyme assays and sensors for wound pathogens such as Pseudomonas aeruginosa (P. aeruginosa), Serratia marcescens, and Escherichia coli have been designed, and are described, for example, in International Patent Application Number PCT/US03/03172, the entire teachings of which are incorporated herein by reference. These protease-based assays use peptides labeled with two dyes typically used in fluorescence resonance energy transfer (FRET) experiments. The non-fluorescent dye DABCYL (4[[4-(dimethylamino)phenyl]azo]benzoic acid) quenches the fluorescent dye EDANS (5-[2-aminoethylamino]naphthalene sulfonic acid) because it is within 100 Å when bound on each end of the peptide substrate. Upon hydrolysis by specific pathogens, the spatially dependent quenching is removed and the EDANS dye emits a bright fluorescence at 490 nM.

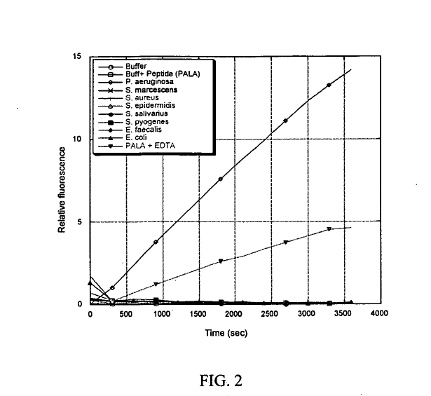

These peptide probes have been used in assays for the inhibition of wound pathogen protease activity by protective proteins. An elastase substrate, PALA 1 (Dabcyl-KHLGGGALGGGAKE-Edans (SEQ ID NO: 11), was used to detect the presence of P. aeruginosa in a sample. To demonstrate the presence of P. aeruginosa in a sample, P. aeruginosa, S. marcescens, S. aureus, S. epidermidis, S. salivarius,. S. pyogenes, E. faecalis, and E. coli bacterial samples were grown in an incubator overnight at 37° C. in 5 mL BHI (Brain Heart Infusion) media. The resulting cultures were spun down and the supernatant was collected. The assays were run in 20 mM tris buffer (pH 7.5) with 150 mM NaCl added. The reaction was carried out with 15 μL of supernatant and 3 μL substrate in 100 μL total volume at 37° C. For inhibition studies, the reaction was performed with 10 mM EDTA under standard assay conditions. The reaction was followed at 485 nm on a fluorometric plate reader. The results of the assay (FIG. 2) showed that PALA 1 was a substrate specific for P. aeruginosa, and was not specific for S. pyogenes, E. coli, S. aureus, S. epidermidis, S. marcescens, or S. salivarius, or E. faecalis.

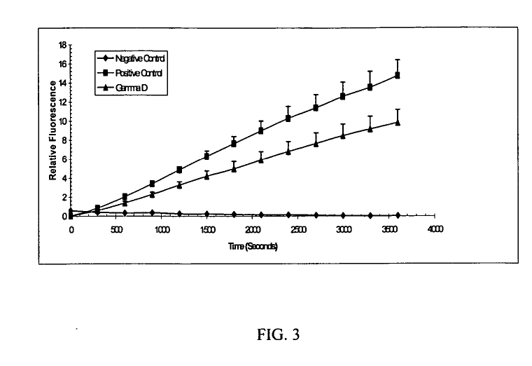

Once the specificity of the assay was established, the ability of γ-D-crystallin to inhibit P. aeruginosa-mediated cleavage of PALA 1 was tested. The PA 14 strain of P. aeruginosa was grown in an incubator overnight at 37° C. in 5 mL Brain Heart Infusion media. The culture was spun down and the supernatant was collected. The assays were run in phosphate buffered saline (PBS) buffer pH 7.5. The reaction was carried out with 15 μL of supernatant, 3 μL of PALA 1 peptide substrate (5 mg/mL) and 50 μg of the γ-D-crystallin protective protein in 100 μL of total volume at 37° C. The PALA1 cleavage reaction was followed on a fluorometric plate reader using an excitation wavelength of 355 nm and an emission wavelength of 485 nm. The positive control was substrate with PA 14 supernatant and the negative control was substrate with buffer. As shown in FIG. 3, this experiment illustrates the protease inhibition fuinction of the γ-D-crystallin protective protein.

An assay was performed to determine the ability of protective proteins α-A-crystallin and Pep/Leu/α-A-crystallin to inhibit elastase activity. 3 μg of recombinant elastase enzyme (CALBIOCHEM® Catalog No. 324676; Darmstadt, Germany) was pre-incubated with either 75 μg of the protective proteins or buffer (negative control) for 20 minutes at room temperature. The ability of the protective proteins to inhibit the elastase activity was measured by the efficiency of the protective protein pre-incubated enzyme to cleave the chromogenic elastase peptide substrate I (MeOSuc-Ala-Ala-Pro-Val-pNA, (SEQ ID NO: 12) CALBIOCHEM® Catalog Number 324696). The results observed using protective proteins α-A-crystallin and Pep/Leu/αα-A-crystallin was consistent- with those described above.

### EXAMPLE 5

### Inhibition of Matrix Metalloprotease (MMP) Using Protective Proteins

To assay for MMP activity in a sample, R&D Systems' (Minneapolis, Minn. enzyme assay for MMPs was employed (according to the manufacturer's instructions, but modified for use of a higher concentration of substrate and a longer incubation time). Fluorogenic Peptide Substrate I (Mca-P-L-G-L-Dpa-A-R-NH2, (SEQ ID NO: 13 ) (ES001, R&D Systems) is a peptide substrate containing a fluorescent 7-methoxycourmarin (Mca) group that is efficiently quenched by energy transfer to the 2,4 dinitrophenyl group of N-3-(2,4-dinitrophenyl)-L-2,3-diaminopropionyl (Dpa). This substrate is used to measure the activity of peptidases that are capable of cleaving the amide bond between the fluorescent group and the quencher group causing an increase of fluorescence that can be monitored in a plate reader. This substrate is an excellent substrate for MMP 1, MMP2, MMP7, MMP8, MMP9, MMP12, MMP13, MMP14, and MMP15; thus, it is a good substrate to measure the general MMP activity of a sample. MMP inhibitors can be used in conjunction with this assay to show that any measured activity is due to MMPs.

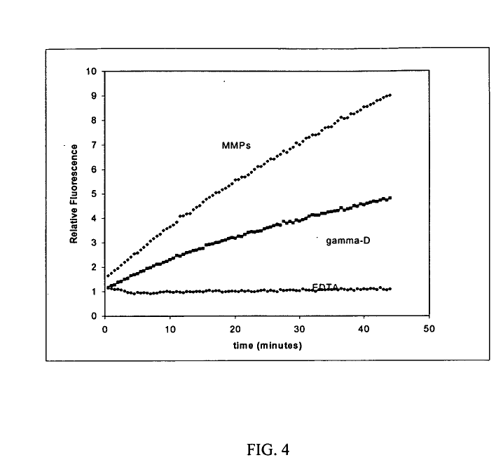

In a study of MMP activity in wounds, porcine wound fluid from a non-infected and an infected wound was used. The porcine wound fluid was collected in an animal study and frozen immediately. The wound fluid was diluted 1:4 in a 50 mM Tris, 150 mM NaCl, 10 mM CaCl₂, pH7.5 buffer. A final concentration of approximately 50 mM MMP substrate (Fluorogenic Peptide Substrate I) was used in each reaction. The non-infected wound fluid showed no activity while the infected wound fluid did show MMP activity. Once the activity of the infected wound fluid was established, its inhibition with 100 mM EDTA (ethylenediaminetetraacetic acid) was tested. The EDTA showed complete inhibition of the substrate activity indicating that this activity is due to metalloproteinases. Next, a final concentration of 0.4 mg/mL γ-D-crystallin was used to inhibit the activity of the MMPs in the wound fluid. Gamma-D-crystallin was determined to inhibit the activity of MMPs by approximately 50% as shown in FIG. 4. Appropriate controls using substrate in buffer were run and showed no activity. This data shows that y-D-crystallin inhibits MMP activity in chronic wound fluid and that protective proteins can be used in healing chronic wounds.

### EXAMPLE 6

### Inhibition of Recombinant Human MMP Activity Using Protective Proteins

Inhibition of the activity of specific human MMPs can be tested using the above-described assay. The ability of the protective proteins to inhibit the activity of MMP2 and MMP9 (purchased from R&D Systems in pro-human recombinant form; Catalog No. 902-MP-010, 911-MP-010) will be tested first. The enzymes can be activated using the recommended APMA (p-aminophenylmercuric acetate) procedure (Sellers et al. (1977) Biochem. J., 163:303-307). The specific activity for both MMP2 and MMP9 will be measured by R&D Systems using the Fluorogenic Peptide Substrate I. Ten μM of Fluorogenic Peptide Substrate I in 50 mM Tris, 150 mM NaCl, 10 mM CaCl₂, and 0.05% Brij can be used with 100 ng of active recombinant human MMP2 and MMP9 in 100 μL final volume to establish their specific activity with this substrate. A fluorescent plate reader can be used to measure the relative fluorescence of the Fluorogenic Peptide Substrate I.

Once the activity is established, the ability of the protective proteins, α-A-crystallin, Pep/Leu/α-A-crystallin, γ-D-crystallin, p26, and SicA to inhibit the activity will be tested. Purified samples of each protective protein for use in this assay can be prepared. Protective proteins can be added individually to active MMP2 and MMP9 samples. Based on prior experiments, it is expected that 50 μg/100 μL (per well) of each protective protein will be sufficient to inhibit MMP activity using the protective proteins. As a positive control, 100 mM ETDA will be used to inhibit the MMP activity. Recombinant human TIMP2 (Catalog No. 971-TM) from R&D Systems, a MMP-specific inhibitor will be used as a control. As described herein, TIMPs (tissue inhibitors of metalloproteases) are a family of proteins that regulate the activation and proteolytic activity of MMPs. There are 4 members of the family (TIMP1, TIMP2, TIMP3, and TIMP4) that are available for use as positive controls from R&D Systems. The ability of the TIMPs to inhibit MMPs can be measured by R&D Systems using the Fluorescent Peptide Substrate I.

For the protective proteins that show inhibition of MMPs, a dose response study will be performed to determine the lowest concentration of protein that is necessary to inhibit MMP activity. Assays will be performed in triplicate and appropriate statistical analysis will be performed on the data. The data obtained from the active protective proteins can be compared to the inhibition of MMPs by the known inhibitors (EDTA and TIMPS).

### EXAMPLE 7

### Inhibition of MMP Activity in Porcine Wound Fluids

Wound fluid from the porcine wound fluid model described above (from infected control wounds and non-infected control wounds) will be used as samples to test the ability of protective proteins to inhibit MMP activity. Fresh wound fluid will be collected using 50 μL of PBS solution applied to the wound bed and aspirated, and frozen immediately (−20° C.). The active MMPs in the samples will be measured and the pro-enzyme forms of the MMPs will not be activated or measured for this study. Since the Fluorogenic Peptide Substrate I assay measures the MMP activity from several different MMPs, there will be no single positive control for the assay. MMP2 and MMP9 can be purchased from R&D Systems in pro-human recombinant form (Catalog No. 902-MP-010, 911 -MP-010), used as positive controls, and are activated as described above.

Overall MMP activity in wound fluid will be measured and then the ability of the protective proteins to inhibit the activity can be determined. An aliquot of the wound fluid will be removed from the -80° C freezer when used in an assay. In general, wound fluid from the same wound will be used for each screening of protective proteins. The MMP activity in the chronic wound fluid sample will be tested to verify that there are detectable levels of activity. MMP activity from fluid from the same wound will then be inhibited with 100 mM EDTA to prove that the activity measured is due to MMPs. In former experience, MMPs levels in non-infected porcine wounds have been very low. These samples can then be used for comparison to the chronic wounds. The ability of the protective proteins p26, and α-A-crystallin, γ-D-crystallin, and SicA to inhibit MMP activity in the chronic wound fluids can then be tested. Protective proteins can be added individually to wound fluid samples from the same wound as described above. Based, on prior experiments, it is expected that 50 μg/100 μL of each protective protein is sufficient to inhibit MMP activity with this initial screen of the protective proteins. For the protective proteins that show inhibition of MMPs. a dose response study will then be performed to determine the lowest concentration of protein necessary to inhibit MMP activity. Assays will be performed in triplicate and appropriate statistical analysis will be performed on the data.

### EXAMPLE 8

### Inhibition of MMP Activity by Protective Proteins in Human Wound Fluid

Fresh frozen wound dressings from anonymous patients presenting chronic wounds can be obtained and tested for the ability of protective proteins to inhibit MMP activity. Using an extraction method to isolate the proteins from the dressings, the activity of MMPs will be analyzed. Dressings from patients with chronic wounds can be placed (using sterile forceps or sterile gloves) in sterile bags and frozen within 30 minutes upon removal from the wound. At the time of the assay, the frozen dressings will be placed with sterile forceps in 50 mL conical tubes and soaked with 1 mL of extraction buffer (0.1 M-Tris/HCl, pH 7.4 containing 0.1% HECAMEG) for 1 hour. After extracting the proteins from the dressing, the fluid will be transferred to sterile 1.5 mL tubes (50 μL aliquots) and frozen in a −80° C. freezer. The level of dilution of the wound fluid will be kept to a minimum and taken into consideration in performing the assay.

The same MMP assay format as described above for the porcine wound fluid will follow. Protective proteins p26, α-A-crystallin, γ-D-crystallin, and SicA will be tested. Protective proteins can be added individually to wound fluid samples from the same wound that tested positive for MMP activity with the Fluorogenic Peptide Substrate I assay. Assays will be performed in triplicate and appropriate statistical analysis will be performed on the data.

### EXAMPLE 9

### Model for Stabilization of Chronic Wound Fluids by Protective Proteins

It has been previously determined that protective proteins that have folding activity (such as SicA, p26, and α-B-crystallin) are able to prevent unfavorable protein intermediates in crude protein extracts. These studies can be extended by examining whether these protein “foldases” can stabilize chronic wound fluid by preventing protein aggregation. The wound fluid will be extracted from the dressings as described above and diluted to an OD₆₀₀ of 0.2 and then incubated at 60° C. in the presence and absence of protective proteins. At time intervals of 0, 10, 20, 40, and 60 minutes, an aliquot will be removed and the level of aggregation measured at 600 nm. The wound fluid will become cloudy in the absence of protective protein but remain clear in the presence of these proteins, thereby demonstrating the level of foldase activity for each protective protein. The results of this study will provide in vitro evidence of the ability of protective proteins to inhibit recombinant human MMP activity as well as MMP activity in chronic wound fluid.

### EXAMPLE 10

### Protective Proteins Provide Cell Protection Under Conditions of High Temperature

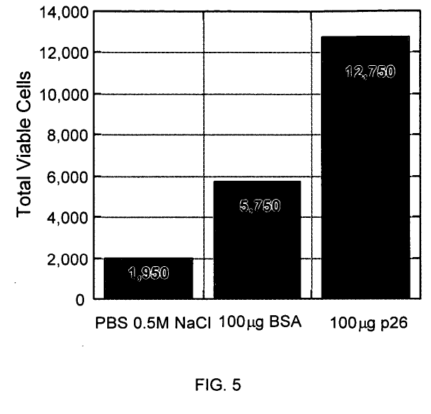

Further in vitro data shows that protective proteins help to stabilize living cells exposed to harsh conditions such as bums and chronic wounds, thereby preventing programmed cell death (apoptosis). Cells heated at 45° C. in the presence of the protective protein p26 were found to remain viable, whereas cells heated in the absence of protective protein lost 85% of their viability, as shown in FIG. 5. Tissue culture cells (normal human astrocytes) were incubated in a humid CO₂ chamber at 45° C. for 30 minutes with PBS, BSA, or p26. Cells were allowed to recover for 1.5 hours in a 37° C. humid CO₂ chamber after adding fresh media. Cells were then trypsinized, spun down, and stained with trypan blue. A hemocytometer was used to count cells. As shown in FIG. 5, the surviving cell count in the presence of p26 protective protein was significantly higher (12,750) than cells in bovine serum albumin (5,750) or in buffered saline (1,950). These results demonstrate that protective proteins promote (enhance) cell viability under harsh conditions. Such protective proteins can be used to treat tissue exposed to harsh conditions, such as burns and chronic wounds.

### EXAMPLE 11

### Comparison of the Benefit of Protective Proteins in Healing Infected Chronic Wounds with Wound Healing Products Currently on the Market in a Porcine Model

The therapeutic properties of the protective proteins were compared with two currently commercially available approaches for healing chronic wounds (PROMOGRAN® and human PDGF BB, both products of Johnson and Johnson, Corp., New Brunswick, N.J.). PDGF BB was tested in the REGRANEX® gel form purchased for these studies (Henry Schein, Mellville, N.Y.). The protective proteins were purified by size exclusion chromatography followed by quaternary amine anion exchange. The protective proteins form large oligomers (˜20 mers) that elute in the void volume of a Sephacryl S-300 column. The fractions containing the correct protein were verified by SDS PAGE, pooled, and then run on a 5 mL QAE HiTrap column. The QAE resin concentrated the protein and removed any trace impurities from the gel filtration step. The fractions from the QAE column containing pure protective protein were pooled and then dialyzed into PBS for the animal study. After dialysis, the protein was stored frozen in 100 mL aliquots at 3-5 mg per mL based on a Bio-Rad protein assay.

Two domestic crossbred swine (female) provided sufficient data to evaluate the effect of the protective proteins on wound healing. The infected partial thickness pig model was studied to determine whether the protective proteins accelerated healing in infected (chronic) wounds. The study was carried out as follows: Identical partial thickness wounds are placed in the back of two female domestic pigs (14 per pig, 7 per side). The partial thickness skin excisional model has been established in the literature in both the guinea pig and the pig model (LeGrand et al. (1995) Wounds, 7:78-89; and Breuing et al. (1997) Plast Reconstr Surg., 100(3):657-664). The wounds were separated from each other to avoid wound-to-wound interference (approximately 3 cm). After the wounds were created, the sites were covered with saline-moistened gauze sponges. The wounds were patted dry, and the pigs were moved to the recovery room prior to application of inoculum.

Each wound was inoculated with 50 μL (containing 10⁵ CFU, measured by the OD at 550 nm) of a Pseudomonas aeruginosa culture. After 24 hours, the test articles were administered topically to the surface of each wound according to Table 2. Protective protein 1 was heat gamma, Protective protein 2 was α-crystallin C domain (truncated α-crystallin) (SGPKIPSREEKPSSAPSS; SEQ ID NO: 34), Protective protein 3 was chimeric heat gamma-α-crystallin C domain (MYKMYMYEEHSREEKPSSAP SS; SEQ ID NO: 35).

Each wound treatment was tested in quadruplicate and randomized. Fifty microliters of 1 mg/mL protective protein solutions, or BSA in phosphate buffered saline (PBS) (negative control) were applied to the appropriate wounds following inoculation with the bacterial culture. REGRANEX® was applied as a gel according to the directions. PROMOGRAN® was applied as a dressing.

TABLE 2
Pig 1: Without Infection (PBS)

| | | | | | | | | |
|---|---|---|---|---|---|---|---|---|
| Cranial | Protector1 | Protector 2 | Protector 3 | BSA | Promogran | Protector 3 | Regranex | Caudal |
| | | | | PBS | | Regranex | | |
| | BSA | Promogran | Protector 3 | Regranex | Protector 1 | Protector 2 | Protector 3 | |
| | PBS | | Regranex | | | | | |

TABLE 3
Pig 2: With Infection (Pseudomonas aeruginosa, 10⁵ CFU)

| | | | | | | | | |
|---|---|---|---|---|---|---|---|---|
| Cranial | Protector 2 | Protector 3 | Protector 1 | Promogran | Regranex | BSA | Protector 3 | Caudal |
| | | | | | | PBS | Regranex | |
| | Promogran | Regranex | Protector 3 | Protector 3 | Protector 2 | Protector 1 | BSA | |
| | | | Regranex | | | | PBS | |

The top of each wound was then covered with a barrier film to avoid cross-contamination due to wound fluid leakage. Sterile gauze was placed over the film and taped to the pig. Each day after surgery (day 0) the dressings were changed and the wounds were photographed and scored for the following parameters: inflammation, edema, erythema, exudate, wound bed, color, induration, odor, and measurements for reepithelialization. The distance to closure and percentage re-epithelialization were calculated based on measurements of the wound area each day.

To aid in dressing change and subsequent wound evaluation, the animal was placed into a sling and anesthetized. Once anesthetized, the dressings were carefully removed using forceps. If exudates and necrotic tissue were present, debridement was performed on each wound using a sterile gauze pad with gentle pressure. All dressings were placed (using sterile forceps or sterile gloves) in sterile bags, labeled with wound number and date, and frozen within 30 minutes upon removal from the wound. Frozen dressings were assessed for the activity and inhibition of MMPs using the Fluorogenic Peptide Substrate I as described herein. Before dressings were re-applied each day, 50 μL of 1 mg/mL protective protein solutions or BSA (negative control) were applied to the appropriate wounds. REGRANEX® was applied as a gel and PROMOGRAN® was applied as a dressing.

At the conclusion of the study, the wound scores for each wound were compiled into a relative healing score. The number of healed wounds based on histology was also determined. Independent t-test and Fisher's Exact test (two-tailed) were used for a statistical comparison of these results. Values of p<0.05 are considered statistically significant. Quantitative histology data sets are subject to statistical testing using the above tests or statistical software such as Statview or JMP, SAS Institute, Cary, N.C.

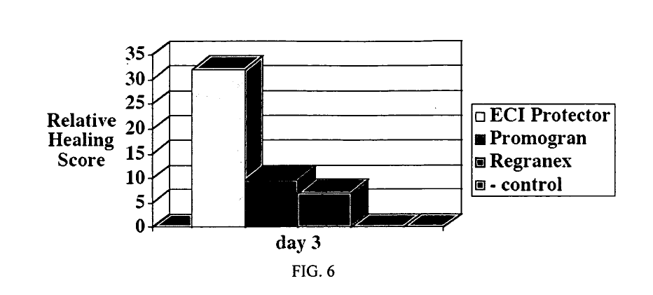

The partial thickness wounds infected with 10⁵ Pseudomonas were examined for 5 days during the treatment with protective proteins, PROMOGRAN® and REGRANEX® and BSA/PBS (control). Data was analyzed in quadruplicate. Protective protein 1 received the highest wound healing score and had an even layer of epidermis based on H&E staining. The control had epidermal necrosis. PROMOGRAN® and REGRANEX® performed similar to the control. FIG. 6 illustrates a graph of the relative healing scores on day 3 of the test.

### EXAMPLE 12

### In vitro Corneal Infection Model

As described herein, a specific and sensitive assay for the detection of P. aeruginosa proteases and elastases has been developed. To test the efficacy of the peptide substrates to detect proteases and elastases under in vivo corneal infection conditions, an in vitro bovine corneal infection model has been developed.

Briefly, fresh bovine corneas were obtained from Animal Technologies (Tyler, Tex.) and stored at 4° C. in Hanks Balanced salt solution (HBSS). Prior to Pseudomonas infection, the corneas were incubated in HBSS supplemented with ampicillin (100 μg/mL) and kanamycin (30 μg/mL) for 1 hour. The corneas were then removed and rinsed three times in PBS to remove any trace antibiotics. These steps were administered so as to prevent contamination of the assay by any preexisting microorganisms. To start infection, an overnight grown culture of P. aeruginosa was diluted in PBS to give a starting inoculum of 100 CFU/mL. The rinsed corneas were placed in 1 mL bacterial suspension (100 CFU/mL) and incubated overnight (18 hours, 37° C.) in aerobic conditions. Corneas incubated in PBS served as a control in these experiments. Assessment of the corneas after overnight incubation with P. aeruginosa revealed the presence of a biofilm characterized by green coloration. No growth was observed in the uninfected corneal control.

The only source of nutrition for the infective Pseudomonas was the cornea, since the dilution of the culture was performed solely in PBS. These results are in agreement with the previous reports of limited nutritional requirement by this pathogen and its ability to adapt to various ecological conditions.

### EXAMPLE 13

### Protease Assay of the Infected Corneal Culture

To determine whether protease activity can be detected in an in vitro bovine corneal infected Pseudomonas culture, protease assays were performed as described herein using the corneal infected culture. Briefly, 5 μL of the infected and uninfected corneal culture was incubated with 3 μL of the protease (PAPA1; Edans-KAAHKSALKSAE-Dabcyl; SEQ ID NO: 25) or elastase (PALA1; Dabcyl-KHLGGGALGGGAKE-Edans; SEQ ID NO:,26) substrate in 100 μL of total volume. The reaction was followed on a 96 well microtiter plate reader for a period of 1 hr at 37° C.

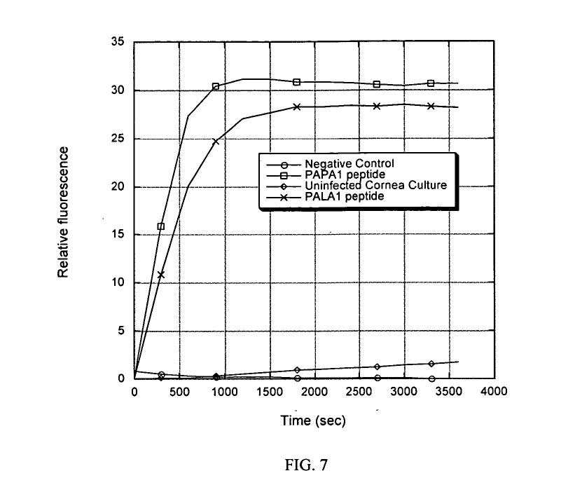

As shown in FIG. 7, protease specific activity was detected using the infected corneal culture. No protease activity was detected when the uninfected (PBS control) corneal culture was used in the assay, indicating that the protease activity observed was P. aeruginosa specific. Further evidence that the substrate PAPA1 is specific for P. aeruginosa was shown as follows. P. aeruginosa, S. marcescens, S. aureus, S. epidermidis, S. salivarius, S. pyogenes, E. faecalis, and E. coli bacterial samples were grown in an incubator overnight at 37° C. in 5 mL BHI (Brain Heart Infusion) media. The resulting cultures were spun down and the supernatant was collected. The assays were run in 20 mM tris buffer (pH 7.5) with 150 mM NaCl added. The reaction was carried out with 7 μL of bacterial supernatant and 3 μL PAPA1 substrate in 100 μL total volume at 37° C. The reaction was followed at 485 nm on a fluorometric plate reader. The results of the assay (FIG. 8) showed that PAPA1 was a substrate specific for P. aeruginosa, and was not specific for S. pyogenes, E. coli, S. aureus, S. epidermidis, S. marcescens, S. salivarius, or E. faecalis.

### EXAMPLE 14

### Demonstration of the Ability of Protective Proteins to Inhibit the Protease Activity of P. aeruginosa

Based on the understanding of the protective proteins described herein, together with the knowledge of the crucial role of MMP and elastase in Pseudomonas-induced keratitis, it is reasonable to expect that the protective proteins will inhibit the activity of the proteolytic enzymes secreted by P. aeruginosa. As described herein several lines of evidence suggest a direct role of proteases such as elastases and MMPs in Pseudomonas-induced microbial keratitis. Pseudomonas proteases in addition to degrading the corneal matrices also play an important role in the evasion of the host immune system via degradation of a number of biologically important proteins. Studies reported by several investigators on targeting of proteases have shown a delay in the progression of the corneal destruction in Pseudomonas keratitis. These studies suggest that the inhibition of these virulence factors has therapeutic application.

The ability of the protective proteins to inhibit P. aeruginosa supernatant-mediated degradation of the peptide substrates is evaluated in this study. The peptide substrates for both proteases (PAPA 1) and elastase (PALA 1) are used in the inhibition assays described below.

### EXAMPLE 15

### Inhibition Assays with P. aeruginosa Grown in BHI Media.

The PA14 strain of P. aeruginosa will be grown in an incubator overnight at 37° C. in 5 mL BHI (Brain Heart Infusion) media. The culture will be spun down and the supernatant collected. The assays will be run in phosphate buffered saline (PBS) buffer (pH 7.5). The reaction will be carried out with 5 μL of spun down supernatant, 3 μL of either protease (PAPA1) or elastase (PALA1) peptide substrates (5 mg/mL) and 50 μg of the protective proteins in 100 μL of total volume at 37° C. Based on prior experiments, it is expected that 50 μg/100 mL of each protective protein will be sufficient to inhibit protease activity in the supernatant. A protease inhibitor cocktail consisting of phenylmethylsulfonylfluoride (PMSF), aprotinin, and pepstatin will serve as an internal control. PBS supplemented with 10 mM EDTA will serve as an internal control when the elastase-specific peptide is used in the inhibition assays. The peptide cleavage reaction will be followed on a fluorometric plate reader using an excitation wavelength of 355 nm and an emission wavelength of 485 nm. For the protective proteins that show inhibition of proteases (MMPs or elastases), a dose response study will be performed to determine the lowest concentration of protein that is necessary to inhibit protease activity. Assays will be performed in triplicate and appropriate statistical analysis will be performed on the data.

### EXAMPLE 16

### Inhibition Assays with P. aeruginosa Infected Bovine Corneal Culture

A similar set of inhibition assays will be performed using the supernatants from infected bovine corneal cultures. Briefly, antibiotic pretreated and rinsed bovine corneas will be infected under sterile conditions with 100 CFU of P. aeruginosa. The infection will be allowed to continue overnight (18 hours, 37° C.) under aerobic conditions. Inhibition assays will then be performed using the same procedures as described above. The corneal culture will then be spun down and assays performed with 5 μL of the supernatants. Protective proteins; α-A-crystallin, γ-D-crystallin and the chimeric protein, and Pep/Leu/α-A-crystallin will be tested individually for their ability to inhibit the proteinase activity of the infected culture supernatant using PAPA1 (protease substrate) and PALA1 (elastase substrate) peptides. Assays will be performed in triplicate and appropriate statistical analysis will be performed on the data.

### EXAMPLE 17

### Demonstration of the Ability of Protective Proteins to Inhibit the P. aeruginosa Mediated Cytopathological Effect (CPE) by Using in Vitro CPE Assays

P. aeruginosa strains have been grouped into three different phenotypes; invasive, cytotoxic and neither invasive nor cytotoxic (Zhu et al. (2002) J Med. Microbiol. 51:1063-70). An inverse correlation exists between invasiveness and cytotoxicity (Fleiszig et al. (1996) Infect. Immun. 64:2288-94). Studies by Wilcox et al. (Zhu et al. (supra) have shown a high level of both protease and elastase activity with the invasive strains of P. aeruginosa as compared to cytotoxic strains. Although, the exact mechanism of P. aeruginosa induced CPEs is not fully understood, indirect evidence suggest the involvement of extracellular proteases. Studies by Callaghan et al. have shown that P. aeruginosa strains that produce a serine protease, Protease IV, were damaging to both the rabbit and mouse corneas, suggesting a role of these proteases in the CPE mediated by P. aeruginosa (Engel et al (1998) J Invest. Ophthalmol. Vis. Sci. 39(3):662-665). Furthermore, studies performed in Acanthamoeba have implicated a direct role of a contact dependent metalloproteinase and three serine proteinases in the cytopathogenic mechanism of this parasite (Cao et al. (1998) J Biol. Chem. 273:15838-45). Based on the understanding of the multitude of extra cellular proteinases secreted by Pseudomonas, together with the knowledge of the destructive capabilities of these enzymes on the corneal epithelial cells it is reasonable to believe that the protective proteins can inhibit the cytopathogenic and cytotoxic events by inhibiting the P. aeruginosa proteinases.

To demonstrate the ability of protective proteins to inhibit the CPE and cytotoxicity mediated via the proteinases, cytopathic and cytotoxic assays using both the invasive and cytotoxic strains of P. aeruginosa will be performed. An invasive microbial keratitis (MK) P. aeruginosa strain, GSU 3, isolated from a human corneal ulcer (Miller et al. (1991) Arch. Ophthalmol. 109:1447-8) can be been obtained. A cytotoxic isolate of P. aeruginosa can also be obtained. These two strains will be used in the in vitro cytopathic and cytotoxic assays using cell cultures of corneal epithelium as target cells. Due to the limited life span of the primary cultures of corneal epithelium, cytopathic assays will be performed using immortalized rabbit corneal epithelial.

To evaluate the P. aeruginosa-induced CPE on corneal epithelial cells, the Pseudomonas strains will be prepared as per the published protocol of Pier et al. (Fleiszig et al. (1996) Infect. Immun. 64:2288-94). The CPE assay will be performed using a modification of the CPE protocol published by Panjwani et al. (Cao et al. (1998) J. Biol. Chem. 273:15838-45). Briefly, both the cytotoxic and invasive strains will be resuspended into Ham's F-12 medium to an inoculum of 10⁶/mL. Triplicate wells of the immmortalized epithelial cells will be incubated with 200 μL of the P. aeruginosa strain to be tested. The plates will be incubated at 37° C. in a CO₂ incubator and periodically examined under a phase contrast microscope for the presence of cell-free plaques in the monolayer for up to 28 hrs. One set of wells will not be inoculated with bacteria and serves as control wells. To evaluate the effect of the protective proteins on P. aeruginosa-induced CPE, the assays will be performed with 50 μg of individual protective proteins. To estimate the relative inhibitory potency of each protective protein, the CPE assay will be terminated when 50% destruction of the monolayer has occurred in the P. aeruginosa incubated wells, based on the observation under a phase-contrast microscope. Following incubation, the cultures will be stained with Giemsa (Diff-Quik, Dade Diagnostic Inc., Aguada, Puerto Rico). Cell densities can be determined as per the published protocol (Cao et al., supra) and CPE expressed as percentage using the following equation:

$$\% CPE = \frac{O . D of epithelial cells incubated with P . aeruginosa}{O . D . of control cells} \times 100$$

The percentage CPE of the protective protein incubated well will also be calculated using the above mentioned formula.

A trypan blue exclusion assay will be performed to evaluate the ability of the protective protein to inhibit P. aeruginosa mediated cytotoxicity. Both the invasive and cytotoxic strain can be used in this experiment and the assay performed as per the protocol of Pier et al. (Fleiszig et al., supra). Briefly, the corneal epithelial cells will be incubated with the bacteria and protective proteins along with appropriate controls as mentioned above and trypan blue will be added to each well. The percentage of cells that take up the stain will be determined by microscopic examination, and a cytotoxicity score given based on the percentage of stained cells.

Both of the above described assays will be performed in triplicates and blinded from the investigator. Spearman rank correlation coefficient test will be used to determine the statistical significance of the assays.

### EXAMPLE 18

### Development of a Synthetic Gamma-D-Crystallin Protective Protein Having High Heat Stability

A synthetic molecule of γ-D-crystallin that has high heat stability was designed. by making conservative amino acid substitutions of the γ-D-crystallin protective protein of SEQ ID NO: 9 with amino acids that have a high heat capacity (absolute entropies) as described by Hutchens et al. (supra). One high heat stable crystallin protective protein has the sequence shown in FIG. 10 (SEQ ID NO: 15). FIG. 11 illustrates a graph of the capacity differential between high heat stable protein (SEQ ID NO: 15) and native human γ-D-crystallin (SEQ ID NO: 9).

The heat stability of human γ-D-crystallin is approximately 37° C. whereas the theoretical heat stability of the newly synthesized high heat stable crystallin was determined to be 57° C. The newly synthesized high heat stable crystallin sequence has 279 proteolytic sites as compared with 237 sites in the original human γ-D-crystallin sequence. Furthermore, protein database sequence analysis (National Center for Biotechnology Information) of synthetic high heat stable crystallin recognized a crystallin-like protein (gamma D crystalline GenBank Accession No. gi|13377002|ref|NP_{—}008822). These findings suggest that this synthetic paralog (e.g., derivative) of human y-D-crystallin is more heat stable. Therefore, it is reasonable to believe that the synthetic high heat stable crystallin can be used as a protease inhibitor in circumstances involving high heat.

### EXAMPLE 19

### Inhibition of MMP Activity by Protective Proteins in Porcine Aortic Valves

Fresh porcine hearts were obtained overnight on ice. The aortic valves with a small portion of aorta were surgically removed from each heart. Two aortic valves with aorta were minced and subsequently extracted overnight at 37° C. Two extraction solutions were used; the first was 50 mM Tris buffer and the second was 150 mM NaCl. One milliliter of solution was used per gram of minced tissue. After incubation overnight, the samples were spun down to remove the majority of the tissue. The samples were then spun through a non-binding 0.45 mm spin filter to remove the remaining solids.

The supernatants were tested for MMP activity using a modified enzyme assay obtained from R&D Systems (Minneapolis, Minn.) and with heat gamma alone. Fluorogenic Peptide Substrate I (Mca-P-L-G-L-Dpa-A-R-NH₂, ES 001, R&D Systems) is a peptide substrate containing a fluorescent 7-methoxycourmarin (Mca) group that is efficiently quenched by energy transfer to the 2,4 dinitrophenyl group of N-3-(2,4-dinitrophenyl)-L-2,3-diaminopropionyl (Dpa). Fluorogenic Peptide Substrate I is useful for measuring the activity of peptidases that are capable of cleaving the amide bond between the fluorescent group and the quencher group. This cleavage causes an increase of fluorescence that can be monitored with a fluorescent plate reader. Fluorogenic Peptide Substrate I is suitable for measuring the general MMP activity of a sample as it has been shown to be cleaved by MMP1, MMP2, MMP7, MMP8, MMP9, MMP12, MMP13, MMP14, and MMP15.

20 μl of Fluorogenic Peptide Substrate I was diluted into 280 μl of 50 mM Tris, 150 mM NaCl, 10 mM CaCl₂, and pH 7.5 buffer. It was found that 20 μl of this substrate stock was sufficient to measure MMP activity in a sample comprising 15 μl of heart valve extract (either Tris or NaCl) and 50 μl of PBS buffer pH 7.0. Activity was measured over about one hour on a fluorescent plate reader with an excitation wavelength of 355 nm and an emission wavelength of 405 nm.

Once the MMP activity of the heart valve extracts was established, a novel heat gamma peptide was used to inhibit the activity of the Tris extract. Heat gamma (HG) comprises a 30 amino acid sequence (MYKMYMYEEHYMYYHHYEMYYEHMYMYMYM; SEQ ID NO: 32) and is a high heat stable protein. Sample activity was inhibited by adding a mixture of 20 μl of Fluorogenic Peptide Substrate I stock solution (as described above), 15 μl of heart valve extract (either Tris or NaCl), 30 μl of heat gamma (2.5 mg/ml), and 35 μl of PBS buffer pH 7.0. FIG. 12 shows the relative fluorescence of both the inhibited sample containing the Tris heart valve extract with heat gamma and a control sample comprising 30 μl of the heat gamma buffer alone. No activity was shown in samples comprising other controls (e.g., Fluorogenic Peptide Substrate I in the different buffers and heat gamma alone). The MMP activity of the Tris heart valve extract is shown as the Protective protein Control (no inhibition) in FIG. 12, and this activity was almost completely inhibited by the heat gamma added to the assay (the “Protective protein” curve). Similar data was obtained for the NaCl extract (not illustrated in FIG. 12).

### EXAMPLE 20

### Inhibition of Porphyromonas gingivalis

P. gingivalis is one of the major etiological agents of adult periodontal disease. Several virulence facts have been identified, including proteinases and adhesion factors. The majority of the P. gingivalis proteinases are collagenases and trypsin-like enzymes, with the majority of the trypsin-like enzymes being cysteine proteases with cleavage specificity after lysine or arginine residues. These cysteine proteases are commonly referred to as “gingipains.” The arginine specific proteases are referred to as Gingipain R and the lysine specific proteases are referred to as Gingipain K. Lysine specific gingipain is encoded by a single gene, kgp, while the arginine specific gingipain is encoded by two related genes, rgpA and rgpB.

Purified RgpB (0.15 μg/assay) was incubated with increasing concentrations (5 μl, 10 μl, 20 μl and 30 μl) of Heat Gamma (2 mg/ml) in the presence of 5 μl T2 FRET peptide (5 mg/ml). The inhibition assays were run using 1× PBS (pH 7) in a total volume of 200 μl at 37° C. The reaction was followed on a fluorometric plate reader using an excitation wavelength of 355 nm and an emission wavelength of 485 nm. FIG. 13A includes a bar graph illustrating the resulting activity of RgpB in the presence of incrementally larger concentrations of heat gamma (HG). As the concentration of heat gamma was increased, the resulting activity of RgpB decreased. FIG. 13B includes a bar graph illustrating the resulting inhibition for the same samples. As the concentration of heat gamma was increased, the resulting inhibition of RgpB increaseds.

Purified HRgpA (0.15 μg/assay) was incubated with increasing concentrations (5 μl, 10μl, 20 μl and 30 μl) of Heat Gamma (2 mg/ml) in the presence of5 μl T2 FRET peptide (5 mg/ml). The inhibition assays were run using 1× PBS (pH 7) in a total volume of 200 μl at 37° C. The reaction was followed on a fluorometric plate reader using an excitation wavelength of 355 nm and an emission wavelength of 485 nm. FIG. 14A includes a bar graph illustrating the resulting activity of the gene HRgpA in the presence of incrementally larger concentrations of heat gamma. As the concentration of heat gamma was increased, the resulting activity of HRgpA decreased. FIG. 14B includes a bar graph illustrating the resulting inhibition for the same samples. As the concentration of heat gamma was increased, the resulting inhibition of HRgpA increased.

Purified KgpB (0.15 μg/assay) was incubated with increasing concentrations (5 μl, 10 μl, 20 μl and 30 μl) of Heat Gamma (2 mg/ml) in the presence of 5 μl PAPA1 FRET peptide (5 mg/ml). The inhibition assays were run using 1× PBS (pH 8) supplemented with 50 mM cysteine, in a total volume of 200 ml at 37° C. The reaction was followed on a fluorometric plate reader using an excitation wavelength of 355 nm and an emission wavelength of 485 nm. FIG. 15A includes a bar graph illustrating the resulting activity of KGP in the presence of incrementally larger concentrations of heat gamma. As the concentration of heat gamma was increased, the resulting activity of KGP decreased. FIG. 15B includes a bar graph illustrating the resulting inhibition for the same samples. As the concentration of heat gamma was increased, the resulting inhibition of KGP increased.

### EXAMPLE 21

### Heat Gamma Mediated Inhibition of P. aeruginosa GSU3

FIG. 16 demonstrates the protective protein mediated inhibition of Pseudomonas protease. Briefly, P. aeruginosa GSU3 supernatant (10⁸CFU) was incubated with either 30 μl (60 μg) of Heat Gamma, or with HG autoclaved 1, 2 or 3 times (AHG1, AHG2, AHG3) or with buffer (HG Buffer). The assays were performed using 3 μl peptide (PAPA1) substrate in a total volume of 100 μl with 1× PBS (pH 7.0) at 37° C. The reactions were followed on a fluorometric plate reader using an excitation wavelength of 355 nm and an emission wavelength of 485 nm. The graphs were plotted using the KaleidaGraph software with relative fluorescence on Y-axis and time in seconds on the X-axis. The results indicate that Heat gamma efficiently inhibits the Pseudomonas proteases as indicated by a decrease in relative fluorescence. HG clearly inhibits the protease activity from Pseudomonas. FIG. 17 illustrates a bar graph of the resulting activity for the samples at the last time point.

### EXAMPLE 22

### Heat Gamma Mediated Inhibition of S. aureus Protease

FIG. 18 demonstrates the protective protein mediated inhibition of Staphylococcus aureus protease. Briefly, S. aureus supernatant (10⁸CFU) was incubated with either 30 μl (60 μg) of Heat Gamma, or with HG autoclaved 1, 2 or 3 times (AHG 1, AHG2, AHG3) or with buffer (HG Buffer). The assays were performed using 5 μl peptide (SSP2) substrate in a total volume of 100 μl with 1× PBS (pH 7.0) at 37° C. The reactions were followed on a fluorometric plate reader using an excitation wavelength of 355 nm and an emission wavelength of 485 nm. The graphs were plotted using the KaleidaGraph software with relative fluorescence on Y-axis and time in seconds on the X-axis. The results indicate that Heat gamma efficiently inhibits the S. aureus proteases as indicated by a decrease in relative fluorescence. FIG. 19 illustrates a bar graph of the resulting activity for the samples at the last time point. HG clearly inhibits the protease activity from Staphylococcus aureus.

### EXAMPLE 23

### Heat Gamma Mediated Inhibition of S. marcescens Protease

FIG. 20 demonstrate the protective protein mediated inhibition of Serratia marcescens protease. Briefly, S. marcescens supernatant (˜10⁸ CFU) was incubated with either 30 μl (60 μg) of Heat Gamma, or with HG autoclaved 1, 2 or 3 times (AHG1, AHG2, AHG3) or with buffer (HG Buffer). The assays were performed using 5 μl peptide (CPI2) substrate in a total volume of 100 μl with 1× PBS (pH 7.0) at 37° C. The reactions were followed on a fluorometric plate reader using an excitation wavelength of 355 nm and an emission wavelength of 485 nm. The graphs were plotted using the KaleidaGraph software with relative fluorescence on Y-axis and time in seconds on the X-axis. The results indicate that Heat gamma efficiently inhibits the S. marcescens proteases as indicated by a decrease in relative fluorescence. HG clearly inhibits the protease activity from S. marcescens. FIG. 21 illustrates a bar graph of the resulting activity for the samples at the last time point.

### EXAMPLE 24

### Heat Gamma Mediated Inhibition of Acanthamoeba castelleni Protease

FIG. 22 demonstrate the protective protein mediated inhibition of Acanthamoeba castelleni. Briefly, A. castelleni supernatant (10⁶CFU) was incubated with either 30 μl (60 μg) of Heat Gamma, or with HG autoclaved 1, 2 or 3 times (AHG1, AHG2, AHG3) or with buffer (HG Buffer). The assays were performed using 5 μl peptide (CPI2) substrate in a total volume of 100 μl with 1× PBS (pH 7.0) at 37° C. The reactions were followed on a fluorometric plate reader using an excitation wavelength of 355 nm and an emission wavelength of 485 nm. The graphs were plotted using the KaleidaGraph software with relative fluorescence on Y-axis and time in seconds on the X-axis. FIG. 23 illustrates a bar graph of the resulting activity for the samples at the last time point. The results indicate that Heat gamma efficiently inhibits the A. castelleni proteases as indicated by a decrease in relative fluorescence.

### EXAMPLE 25

### Heat Gamma Mediated Inhibition of MMP 1 Protease

FIG. 24 demonstrate the Heat Gamma (HG) mediated inhibition of matrix metalloproteinase MMP 1: Briefly, 0.2 μg of activated recombinant MMP1 was incubated with 30 μl (60 μg) of Heat Gamma, or with HG autoclaved 1, 2 or 3 times (AHG1, AHG2, AHG3) or with buffer (HG Buffer). The assays were performed using 20 μl Fluorogenic Peptide substrate I in a total volume of 100 μl with 1× PBS (pH 7.0) at 37° C. The reactions were followed on a fluorometric plate reader using an excitation wavelength of 355 nm and an emission wavelength of 405 nm. The graphs were plotted using the KaleidaGraph software with relative fluorescence on-Y-axis and time in seconds on the X-axis. The results indicate that Heat gamma efficiently inhibits the MMP1 activity as indicated by a decrease in relative fluorescence. FIG. 25 illustrates a bar graph of the resulting activity for the samples at the last time point.

### EXAMPLE 26

### Heat Gamma Mediated Inhibition of MMP2 Protease

FIG. 26 demonstrate the Heat Gamma (HG) mediated inhibition of matrix metalloproteinase MMP2. Briefly, 0.2 μg of activated recombinant MMP2 was incubated with 30 μl (60 μg) of Heat Gamma, or with HG autoclaved 1, 2 or 3 times (AHG1, AHG2, AHG3) or with buffer (HG Buffer). The assays were performed using 20 μl Fluorogenic Peptide substrate I in a total volume of 100 μl with 1× PBS (pH 7.0) at 37° C. The reactions were followed on a fluorometric plate reader using an excitation wavelength of 355 nm and an emission wavelength of 405 nm. The graphs were plotted using the KaleidaGraph software with relative fluorescence on Y-axis and time in seconds on the X-axis. FIG. 27 illustrates a bar graph of the resulting activity for the samples at the last time point. The results indicate that Heat gamma efficiently inhibits the MMP2 activity as indicated by a decrease in relative fluorescence.

### EXAMPLE 27

### Heat Gamma Mediated Inhibition of MMP9 Protease

FIG. 28 demonstrate the Heat Gamma (HG) mediated inhibition of matrix metalloproteinase MMP9. Briefly, 0.270 μg of activated recombinant MMP9 was incubated with 30 μl (60 μg) of Heat Gamma, or with HG autoclaved 1, 2 or 3 times (AHG1, AHG2, AHG3) or with buffer (HG Buffer). The assays were performed using 20 μl Fluorogenic Peptide substrate I in a total volume of 100 ml with 1× PBS (pH 7.0) at 37° C. The reactions were followed on a fluorometric plate reader using an excitation wavelength of 355 nm and an emission wavelength of 405 nm. The graphs were plotted using the KaleidaGraph software with relative fluorescence on Y-axis and time in seconds on the X-axis. FIG. 29 illustrates a bar graph of the resulting activity for the samples at the last time point. The results indicate that Heat gamma efficiently inhibits the MMP9 activity as indicated by a decrease in relative fluorescence.

### EXAMPLE 28

### Growth Inhibition of Pseudomonas With Heat Gamma

A BHI agarose plate was inoculated with 100 μl of 10⁸ P. aeruginosa. 100 μl (˜250 μg) of heat gamma was pipetted onto a portion of the surface of the plate, while a similar amount of control buffer was pipetted onto another portion of the plate. The plate was grown overnight at 37° C. A zone of inhibition was clearly visible with the protector protein heat gamma, but no clearance was observed with the buffer control.

### EXAMPLE 29

### Novel Recombinant Protein: Gamodulin

A novel recombinant protein has been derived from gamma crystallin and calmodulin. The protein described herein, gamodulin, comprises the amino terminal domain of gamma crystallin and the calcium binding domain (EF hand) of calmodulin and should reasonably have both the ability to sequester calcium and inhibit proteases.

The clone for human gamma crystallin was purchased from Invitrogen and recloned into a E. coli t7 expression vector using the restriction sites NdeI and Xho I (pET24a, Novagen). Human calmodulin I was cloned by reverse transcriptase using the methods described by the manufacturers. Briefly, RNA was purified from cultured normal human astrocyts using RNA purification kit (FastRNA Pro Red Kit, cat# 6035-050). The RNA was reverse transcribed and amplified using Invitorogen's Superscript kit (One Step RT-PCR system, 10928-034) and the following parameters: -42° C. for about 30 minutes, followed by 94° C. for about 2 minutes, then cycles of 50° C. annealing for 30 seconds, 72° C. extension for about 1 minute, and 94° C. melting for 1 minute.

Primers to link the gamma crystallin to calmodulin have an internal and unique restriction sites, and include: CTA GCT AGC TAG ATG GGG AAG ATC ACC (Gamma Forward; SEQ ID NO: 33); CCC AAG CTT GGG GCC AGG AAC ACA CAG (Gamma Reverse; SEQ ID NO: 29); CCC AAG CTT GGG TTT GAC AAG GAT GGC (Calmodulin Forward; SEQ ID NO: 30); CGC GGA TCC GCG TCA TTT TGC AGT CAT (Calmodulin Reverse; SEQ ID NO: 31). This recombinant protein will be expressed and characterized for its calcium binding activity and protease inhibition activity using the methods described herein, as well as other well-known techniques. For example, the activities of gamodulin on confocal microscope with isolated porcine heart valves and fluorescent and colorometric probes for calcium and protease activities (Fura2 and Flua2, available from Molecular Probes of Eugene, OR, and Alizarin Red, available from Sigma Aldrich of St. Louis, Mo.). Fresh porcine hearts will be obtained from Animal Technologies (Austin, Tex.). Just prior to use, the valves will be dissected out, removing aortic wall and rinsed with PBS and stored at 4° C. Quantitative measurements of the relative fluorescent and localization of any calcium deposits and foci for proteolytic activity of MMPs are then made.

Calcification and degradation of biosynthetic heart valves is a major problem in cardiac care. Biosynthetic heart valves only last 15-20 years due to proteolysis and calcification. The calcification of the valve leads to stenosis and tearing of the tissue that can lead to ultimate failure of the valve. Gamodulin can sequester calcium and inhibit proteases to combat this problem.

While this invention has been particularly shown and described with references to preferred embodiments thereof, it will be understood by those skilled in the art that various changes in form and details can be made therein without departing from the scope of the invention encompassed by the appended claims.

### EXAMPLE 30

### Porphyromonas gingivalis Assay Development and Dose-Dependent Inhibition

An array of specific enzyme assays and sensors for several pathogens (e.g., Pseudomonas aeruginosa, Serratia marcescens, and E. coli.) These protease-based assays use peptides labeled with two dyes typically used in fluorescence resonance energy transfer (FRET) experiments. The non-fluorescent dye DABCYL (4-[4-(dimethylamino)phenyl] azo benzoic acid) quenches the fluorescent dye EDANS (5-[2-aminoethylamino]naphthalene sulfonic acid) because it is within 100 Å when bound on each end of the peptide substrate. Upon hydrolysis by specific pathogens, the spatially dependent quenching is removed and the EDANS dye emits a bright fluorescence at 490 nm.

In preliminary screenings for peptide substrates, the two peptides PAPA1 and T2 (SEQ ID NO: 36; (Dabcyl)-KVSRRRRRGGD-(Edans)) were identified for detecting the protease activity from P. gingivalis. PAPA1 peptide substrate consists of two lysine residues and can be efficiently cleaved by the lysine gingipain, Kgp.

P. gingivalis was grown under anaerobic condition at 37° C. in BHI media supplemented with hemin and menadiaone. Assays were performed using both the culture (including cells and media) or supernatant in 1× PBS (pH 7.5). The reaction was carried out with 3 ml of peptide substrate and 7 ml of supernatant in a 200 μl total volume at 37° C. The excitation wavelength was 355nm and the reaction was followed at an emission wavelength of 485 nm on a fluorometric plate reader. T2 peptide substrate consists of a stretch of arginine residues and is a good target peptide for arginine gingipains, HRgpA and RgpB. PAPA1 and T2 FRET peptide substrates were shown to be efficiently cleaved with the P. gingivalis supernatant.

### EXAMPLE 31

### Inhibition of proteases from P. gingivalis

The P. gingivalis strain W83 (obtained from Dr. Caroline Genco, Boston University, Boston, Mass.) was grown anaerobically in BHI media supplemented with hemin and menadione under anaerobic conditions at 37° C. 1 ml of culture (OD₅₅₀3.0, approximately 1×10¹⁰ CFU/ml) was spun down and the supernatant collected. The assays were performed in PBS supplemented with 50 mM cysteine (pH 7.5). The reaction was carried out with 7 μl of supernatant (1×10¹⁰ CFU/ml), 3 μl of T2 peptide substrate (5 mg/ml) and different concentrations of HG in 200 μl total volume at 37° C.

The ability of HG to inhibit the proteolytic activity of the P. gingivalis supernatant was measured on a fluorometric plate reader using an excitation wavelength of 355 nm and an emission wavelength of 485 nm. A dose-dependent inhibition of P. gingivalis W83 supernatant was observed with increasing concentration of HG. Assays were performed in triplicates and appropriate statistical analyses were performed. A 70% inhibition was observed at the highest concentration of HG (100 μg) used.

The purified gingipains, HRgpA, RgpB and Kgp (obtained from Dr. Genco) was analyzed in inhibition studies to determine the ability of HG to specifically inhibit the purified gingipains. The FRET assays were performed as previously described with slight modifications. Briefly, 0.148 μg of the purified gingipain (HRgpA, RgpB and Kgp) was incubated with increasing concentration of HG (2.5 mg/ml). The assays were performed using 5 μl of peptide substrate (T2 for HRgpA and RgpB, PAPA1 for Kgp) in a total volume of 200 μl with 1× PBS supplemented with 50 mM cysteine. The reactions were followed on a fluorometric plate reader using an excitation wavelength of 355 nm and an emission wavelength of 485 nm.

HG was observed to efficiently inhibits the activity of arginine gingipains, as indicated by a decrease in relative fluorescence intensity. The percent inhibition was calculated based on the fluorescence intensity at the last time point of the assay. Approximately 90 % and 80 % of the gingipain activity was observed at the highest concentration (75 μg) of HG tested. A dose dependent inhibition was also observed when purified lysine gingipain, Kgp was used in similar assays. A relatively, lower level of inhibition was observed in case of Kgp when compared to that of lysine gingipains (HRgpA and RgpB). A 50% inhibition was observed at the highest concentration of HG (75 μg ) used.

While this invention has been particularly shown and described with references to preferred embodiments thereof, it will be understood by those skilled in the art that various changes in form and details can be made therein without departing from the scope of the invention encompassed by the appended claims.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of inhibiting cell or tissue damage comprising contacting said cell or tissue with
a protective protein;
an active fragment, variant or derivative of a protective protein; or
a chimeric protein comprising a protective protein,
whereby the contact inhibits damage to the cell or tissue.

2. The method of claim 1, wherein said protective protein is selected from the group consisting of p26, SicA, and a crystallin protein.

3. The method of claim 2, wherein said protective protein is selected from the group consisting of α-A-crystallin, α-B-crystallin, and γ-D-crystallin.

4. The method of claim 2, wherein said protective protein is a high heat stable crystallin protein.

5. The method of claim 1, wherein the chimeric protein comprises p26, SicA, or a crystallin protein.

6. The method of claim 1, wherein said cell or tissue damage is pathogen-induced.

7. The method of claim 1, wherein the protective protein, the active fragment, variant or derivative, or the chimeric protein is in a pharmaceutically acceptable carrier.

8. The method of claim 1, wherein the cell or tissue is located in an oral cavity of a mammal.

9. The method of claim 1, wherein the cell or tissue is a portion of a cardiac valve.

10. The method of claim 1, wherein the contact decreases or prevents cationic antimicrobial peptide degradation.

11. A method of inhibiting protease activity in a cell or tissue comprising contacting the cell or tissue with
a protective protein;
an active fragment, variant or derivative of a protective protein; or
a chimeric protein comprising a protective protein,
whereby the contact inhibits protease activity in the cell or tissue.

12. The method of claim 11, wherein said protective protein is selected from the group consisting of p26, SicA, and a crystallin protein.

13. The method of claim 12, wherein said crystallin protein is selected from the group consisting of α-A-crystallin, α-B-crystallin, and γ-D-crystallin.

14. The method of claim 12, wherein said crystallin protein is a high heat stable crystallin protein.

15. The method of claim 11, wherein the chimeric protein comprises p26, SicA, or a crystallin protein.

16. The method of claim 11, wherein the protective protein, the active fragment, variant or derivative, or the chimeric protein is in a pharmaceutically acceptable carrier.

17. The method of claim 11, wherein said protease is a matrix metalloprotease.

18. The method of claim 11, wherein said protease is an elastase.

19. The method of claim 11, wherein the cell or tissue is located in an oral cavity of a mammal.

20. The method of claim 11, wherein the cell or tissue is a cardiac cell or cardiac tissue.

21. The method of claim 11, wherein the contact decreases or prevents cationic antimicrobial peptide degradation.

22. A method of inhibiting the virulence of a pathogen comprising contacting a substance released by said pathogen with
a protective protein;
an active fragment, variant or derivative of a protective protein; or
a chimeric protein comprising a protective protein,
whereby the contact inhibits the virulence of a pathogen.

23. The method of claim 22, wherein said protective protein is selected from the group consisting of p26, SicA, and a crystallin protein.

24. The method of claim 23, wherein said crystallin protein is selected from the group consisting of α-A-crystallin, α-B-crystallin, and γ-D-crystallin.

25. The method of claim 23, wherein said crystallin protein is a high heat stable crystallin protein.

26. The method of claim 22, wherein said protective protein is a chimeric protein comprising p26, SicA, or a crystallin protein.

27. The method of claim 22, wherein the protective protein, the active fragment, variant or derivative, or the chimeric protein is in a pharmaceutically acceptable carrier.

28. The method of claim 22, wherein the contact occurs in an oral cavity of a mammal.

29. The method of claim 22, wherein the contact occurs in the cardiac system of a mammal.

30. The method of claim 22, wherein the contact decreases or prevents cationic antimicrobial peptide degradation.

31. A method of inhibiting proteolysis of a cationic antimicrobial peptide in a cell or tissue comprising contacting said cell or tissue with
a protective protein;
an active fragment, variant or derivative of a protective protein; or
a chimeric protein comprising a protective protein,
whereby the contact inhibits proteolysis of a cationic antimicrobial peptide in the cell or tissue.

32. The method of claim 31, wherein said protective protein is selected from the group consisting of p26, SicA, and a crystallin protein.

33. The method of claim 32, wherein said crystallin protein is selected from the group consisting of (α-A-crystallin, α-B-crystallin, and γ-D-crystallin.

34. The method of claim 32, wherein said crystallin protein is a high heat stable crystallin protein.

35. The method of claim 31, wherein said protective protein is a chimeric protein comprising p26, SicA, or a crystallin protein.

36. The method of claim 31, wherein the protective protein, the active fragment, variant or derivative, or the chimeric protein is in a pharmaceutically acceptable carrier.

37. The method of claim 31, wherein said cationic antimicrobial peptide is selected from the group consisting of a defensin, a cathelicidin, and a thrombocidin.

38. The method of claim 31, wherein the contact occurs in an oral cavity of a mammal.

39. The method of claim 31, wherein the contact occurs in the cardiac system of a mammal.

40. A chimeric protein comprising Pepstatin A, Leupeptin, and an alpha-A-crystallin polypeptide.

41. The chimeric protein of claim 40, wherein said chimeric polypeptide comprises the sequence of SEQ ID NO: 14.

42. The chimeric protein of claim 40, wherein said chimeric polypeptide consists of the sequence of SEQ ID NO: 14.

43. A high heat stable crystallin protein.

44. The protein of claim 43, wherein said protein comprises the sequence of SEQ ID NO: 15.

45. The protein of claim 43, wherein said protein consists of the sequence of SEQ ID NO: 15.

46. The protein of claim 43, wherein said protein comprises the sequence of SEQ ID NO: 32.

47. The protein of claim 46, wherein said protein consists of the sequence SEQ ID NO: 32.
